(19) United States
(12) Patent Application Publication
(10) Pub. No.: US 2025/0213580 A1
(43) Pub. Date: Jul. 3, 2025

(54) Improved Thiopurine Formulation and Treatment Methods

(71) Applicant: Douglas Pharmaceuticals Ltd, Auckland (NZ)

(72) Inventors: Peter William Surman, Auckland (NZ); Zhen Shi, Auckland (NZ); Christopher Ritchie, Auckland (NZ); Timothy Henri Jeremy Florin, Auckland (NZ)

(21) Appl. No.: 18/850,266

(22) PCT Filed: Mar. 24, 2023
(86) PCT No.: PCT/NZ2023/050038

§ 371 Date: Sep. 24, 2024

(30) Foreign Application Priority Data
Mar. 25, 2022 (AU) 2022900759

(51) Int. Cl. A61K 31/52; A61K 9/20; A61K 9/28

(52) U.S. Cl. CPC A61K 31/52; A61K 9/2013; A61K 9/2031; A61K 9/282; A61K 9/2846

(57) ABSTRACT

The present invention relates generally to improved formulations of 6-thioguanine (6-TG), their methods of preparation, and their use in treatment methods. The invention provides a pharmaceutical composition including 6-TG, and a polyethylene oxide polymer having a molecular weight of between about 900,000 g/mol and about 9,000,000 g/mol, their methods of preparation, and their use in methods for treating a disease or condition of the distal intestine that responds to 6-TG, wherein the 6-TG is released in the distal intestine.

### CROSS-REFERENCE TO RELATED APPLICATIONS

This application is the United States national phase of International Application No. PCT/NZ2023/050038 filed Mar. 24, 2023, and claims priority to Australian Provisional Patent Application No. 2022900759 filed Mar. 25, 2022, the disclosures of which are hereby incorporated by reference in their entireties.

### BACKGROUND OF THE INVENTION

### Field of the Invention

The present invention relates generally to improved formulations of 6-thioguanine (6-TG), their methods of preparation, and their use in treatment methods.

### Description of Related Art

Thiopurine compounds 6-mercaptopurine (6-MP; 3,7-dihydropurine-6-thione) and azathioprine (AZA; 6-(3-methyl-5-nitroimidazol-4-yl) sulfanyl-7H-purine) are pro-drugs which are metabolized to form 6-thioguanosine-triphosphate (6-TGTP) as the principal active metabolite via the salvage pathway of purine metabolism. AZA and 6-MP are used in the treatment of a number of diseases and conditions, such as inflammatory conditions; cancers; autoimmune conditions; and post-transplant immunosuppression. These compounds are also used in therapy for inflammatory bowel disease (IBD) despite the long time-to-onset of clinical activity of typically three to four months, and the relatively fine line between the desired clinical end-point of controlled immunosuppression and the side effect of leukopenia.

Conversion of AZA or 6-MP by some cells in the body, including liver cells and white blood cells, produces active metabolites in the form of thioguanine nucleotides (6-TGNs; principally 6-TGTP, and to a lesser extent 6-TGMP and 6-TGDP). This metabolic pathway is summarised diagrammatically in FIG. 1.

6-TGTP acts on activated circulating T-lymphocytes leading to proliferation arrest and then apoptosis. The reduction in gut-homing circulating immune cells then turns off the adaptive immune system's amplification of inflammation at the site of IBD. However, the metabolism of AZA/6-MP to thioguanine nucleotides (6TGNs) is rate limited by the enzymes inosine monophosphate dehydrogenase (IMPDH) and guanosine monophosphate synthetase (GMPS). The appearance of 6-TGNs from the metabolism of AZA/6-MP takes days, with a steady state taking at least about four weeks to achieve. The pharmacodynamic action, i.e., the clinical action, typically takes three to four months to achieve. Furthermore, methylated metabolites such as me6MP, me6MPr and meTIMP are also produced during the conversion of AZA and 6-MP. High levels of these methylated metabolites are associated with a number of undesirable side-effects, including hepatotoxicity, nausea, extreme fatigue, myalgia, pancreatitis, and myelosuppression (beyond that predicted from a 6-TGTP-induced immunosuppressive effect). These side-effects are serious in a large percentage of patients prescribed either 6-MP or AZA and this factor, along with the slow onset of action, limits the use of 6-MP and AZA.

According to current clinical practice, an alternative thiopurine compound, 6-thioguanine (6-TG; 2-amino-6,7-dihydro-3H-purine-6-thione), may be prescribed as an alternative in the situation when 6-MP or AZA therapy has been unsuccessful.

6-TG is converted to TGNs, but is not functionally equivalent to 6-MP or AZA therapy. With reference to FIG. 1, 6-TG is converted to 6-TGTP, the same principal active metabolite as 6-MP or AZA, but 6-TG is metabolised more directly by the enzyme hypoxanthine-guanine phosphoribosyltransferase (HPRT), i.e., the same principal active metabolite formed by metabolism of 6-MP or AZA, but the metabolic pathway is different. 6-TG is first converted to a monophosphate analogue (6-TGMP) by HPRT and is then converted via kinases to the active metabolite 6-TGTP. Fewer metabolic steps are required to convert 6-TG to the active drug, and the conversion of 6-TG is not rate-limited by the enzymes IMPDH and GMPS. Therefore conversion of 6-TG to 6-TGTP is significantly faster than 6-MP or AZA and so a clinically faster onset of action is possible by using 6-TG. Furthermore, major adverse effects associated with methylated metabolites produced during metabolism of 6-MP or AZA do not occur with metabolism of 6-TG.

Unfortunately, the faster onset of 6-TG treatment is strongly associated with serious hepatotoxic side-effects producing liver vascular disease comprising sinusoidal obstructive syndrome (SOS); veno-occlusive disease (VOD); or nodular regenerative hyperplasia (NRH). These vascular hepatotoxic side-effects, which are unusual with 6-MP or AZA, are related to high concentrations of 6-TG in the hepatic portal circulation, particularly when 6-TG is used in high dose.

When 6-TG is administered in an immediate release form (bolus dose) to IBD patients it is converted to 6-TGTP systemically by the liver and white blood cells. It is believed that 6-TGTP acts on circulating white blood cells (in particular, T-lymphocytes) thus mediating the beneficial immunomodulation to treat IBD. 6-TG formulations are usually designed to provide substantial systemic concentrations of 6-TGN to provide the desired immunosuppressive function. In order to minimise the adverse hepatic vascular side-effects, extended release formulations that provide a 3 hour extended release profile of 6-TG have been developed. The majority of 6-TG from these formulations is released in the proximal small intestine to achieve maximum systemic absorption of 6-TG at a controlled rate while minimising toxicity from high hepatic portal Cₘₐₓ.

More recently, it has been discovered that T- and B-lymphocytes are not required for 6-TG to ameliorate colitis. 6-TG can act therapeutically in a manner which is independent of the adaptive immune system. 6-TG can act locally and rapidly at sites of inflammation, and be converted to the active metabolite 6-TGTP by the intestinal mucosa and luminal bacteria without appreciable systemic concentration. A low dose of 6-TG, but not 6-MP results, in a rapid local anti-inflammatory effect in the colon without significant immunosuppression. Furthermore, the targeted release provides a method of treatment of IBD which provides a faster mechanism of action that does not generate appreciable systemic concentrations of 6-TGN, and thus addresses the problems of the toxic side effects of 6-TG, namely vascular hepatotoxicity (such as SOS) and excessive immunosuppression due to rapid anabolic metabolism of 6-TG. This targeted 6-TG administration is efficacious as the active metabolite is generated and released at the site of inflammation. Vascular hepatotoxicity and myelosuppression are minimised as the colon is poorly absorptive and the therapeutic effect is largely through action of a local low dose of 6-TG on the distal intestine.

It has further been demonstrated that intra-rectal administration of 6-TG ameliorated spontaneous colitis in a mouse model within 14 days in the distal colon. There was no improvement in colitis in mid-colon or proximal colon indicating that the beneficial effect of 6-TG was local. Intra-rectal administration of 6-MP did not provide any significant improvement in colitis, demonstrating the lack of functional equivalence of 6-TG and 6-MP/AZA. Local delivery of 6-TG to sites of intestinal inflammation in IBD thus provides significant advantages over current immunomodulating thiopurine therapies.

However, there remain challenges with preparing 6-TG formulations that provide a sufficiently long dissolution time, and which exhibit a zero-order, linear release profile, so as to evenly distribute 6-TG through the distal intestine thereby providing a local anti-inflammatory effect at the site of inflammation.

There is a need for improved formulations that address one or more of the challenges of the prior art.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the invention provides a pharmaceutical composition comprising 6-thioguanine (6-TG), and a polyethylene oxide polymer having a molecular weight of between about 900,000 g/mol and about 9,000,000 g/mol.

In one embodiment, the composition is formulated for oral administration.

In one embodiment, the composition is formulated for release of 6-TG in the distal intestine.

In one embodiment, the polyethylene oxide polymer has a molecular weight greater than about 900,000 g/mol and less than about 9,000,000 g/mol. In one embodiment, the polyethylene oxide polymer has a molecular weight greater than about 900,000 g/mol and less than about 7,000,000 g/mol. In one embodiment, the polyethylene oxide polymer has a molecular weight of between about 1,000,000 g/mol and 6,000,000 g/mol; between about 1,000,000 g/mol and 5,000,000 g/mol; between about 1,000,000 g/mol and 4,000,000 g/mol; between about 1,000,000 g/mol and 3,000,000 g/mol.

In one embodiment, the polyethylene oxide polymer has a molecular weight of about 900,000 g/mol; 1,000,000 g/mol; 1,100,000 g/mol; 1,200,000 g/mol; 1,300,000 g/mol; 1,400,000 g/mol; 1,500,000 g/mol; 1,600,000 g/mol; 1,700,000 g/mol; 1,800,000 g/mol; 1,900,000 g/mol; 2,000,000 g/mol; 2,000,000 g/mol; 2,100,000 g/mol; 2,200,000 g/mol; 2,300,000 g/mol; 2,400,000 g/mol; 2,500,000 g/mol; 2,600,000 g/mol; 2,700,000 g/mol; 2,800,000 g/mol; 2,900,000 g/mol; 3,000,000 g/mol; 3,100,000 g/mol; 3,200,000 g/mol; 3,300,000 g/mol; 3,400,000 g/mol; 3,500,000 g/mol; 3,600,000 g/mol; 3,700,000 g/mol; 3,800,000 g/mol; 3,900,000 g/mol; 4,000,000 g/mol; 4,100,000 g/mol; 4,200,000 g/mol; 4,300,000 g/mol; 4,400,000 g/mol; 4,500,000 g/mol; 4,600,000 g/mol; 4,700,000 g/mol; 4,800,000 g/mol; 4,900,000 g/mol; 5,000,000 g/mol; 5,100,000 g/mol; 5,200,000 g/mol; 5,300,000 g/mol; 5,400,000 g/mol; 5,500,000 g/mol; 5,600,000 g/mol; 5,700,000 g/mol; 5,800,000 g/mol; 5,900,000 g/mol; 6,000,000 g/mol; 6,100,000 g/mol; 6,200,000 g/mol; 6,300,000 g/mol; 6,400,000 g/mol; 6,500,000 g/mol; 6,600,000 g/mol; 6,700,000 g/mol; 6,800,000 g/mol; 6,900,000 g/mol; 7,000,000 g/mol; 7,100,000 g/mol; 7,200,000 g/mol; 7,300,000 g/mol; 7,400,000 g/mol; 7,500,000 g/mol; 7,600,000 g/mol; 7,700,000 g/mol; 7,800,000 g/mol; 7,900,000 g/mol; 8,000,000 g/mol; 8,100,000 g/mol; 8,200,000 g/mol; 8,300,000 g/mol; 8,400,000 g/mol; 8,500,000 g/mol; 8,600,000 g/mol; 8,700,000 g/mol; 8,800,000 g/mol; 8,900,000 g/mol; or 9,000,000 g/mol.

In one embodiment, the polyethylene oxide polymer has a molecular weight of about 900,000 g/mol. In one embodiment, the polyethylene oxide polymer has a molecular weight of about 1,000,000 g/mol. In one embodiment, the polyethylene oxide polymer has a molecular weight of about 2,000,000 g/mol. In one embodiment, the polyethylene oxide polymer has a molecular weight of about 4,000,000 g/mol. In one embodiment, the polyethylene oxide polymer has a molecular weight of about 5,000,000 g/mol. In one embodiment, the polyethylene oxide polymer has a molecular weight of about 7,000,000 g/mol.

In one embodiment, the polyethylene oxide polymer has a molecular weight of about 2,000,000 g/mol.

In one embodiment, the polyethylene oxide polymer comprises two or more different approximate molecular weights.

In one embodiment, the polyethylene oxide polymer comprises substantially a single approximate molecular weight.

In one embodiment, the pharmaceutical composition is a tablet.

In one embodiment, the tablet comprises a core and a coating.

In one embodiment, the tablet comprises about 1 to 50 mg of 6-TG in each tablet. In one embodiment, the tablet comprises about 1 to 40 mg of 6-TG in each tablet. In one embodiment, the tablet comprises about 1 to 30 mg of 6-TG in each tablet. In one embodiment, the tablet comprises about 1 to 20 mg of 6-TG in each tablet. In one embodiment, the tablet comprises about 5 to 20 mg of 6-TG in each tablet.

In some embodiments, the tablet comprises about 1 mg, 2 mg, 3 mg, 4 mg, 5 mg, 10 mg, 20 mg, 25 mg, 30 mg, 40 mg or 50 mg of 6-TG.

In one embodiment the tablet comprises about 10 mg of 6-TG.

In one embodiment, 6-TG is present in an amount of about 1 to 30% w/w of the pharmaceutical composition. In one embodiment, 6-TG is present in an amount of about 1 to 20% w/w of the pharmaceutical composition. In one embodiment, 6-TG is present in an amount of about 1 to 10% w/w of the pharmaceutical composition. In one embodiment, 6-TG is present in an amount of about 1 to 5% w/w of the pharmaceutical composition. In one embodiment, 6-TG is present in an amount of about 2 to 4% w/w of the pharmaceutical composition.

In one embodiment, 6-TG is present in an amount of about 1 to 30% w/w of the core. In one embodiment, 6-TG is present in an amount of about 1 to 10% w/w of the core. In one embodiment, 6-TG is present in an amount of about 2 to about 3% w/w.

In one embodiment, the polyethylene oxide polymer comprises about 60 to 98% w/w of the pharmaceutical composition. In one embodiment, the polyethylene oxide polymer comprises about 65 to 98% w/w of the pharmaceutical composition. In one embodiment, the polyethylene oxide polymer comprises about 70 to 98% w/w of the pharmaceutical composition. In one embodiment, the polyethylene oxide polymer comprises about 80 to 98% w/w of the pharmaceutical composition. In one embodiment, the polyethylene oxide polymer comprises about 85 to 98% w/w of the pharmaceutical composition.

In one embodiment, the polyethylene oxide polymer comprises about 70 to 99% w/w of the core of the tablet. In one embodiment, the polyethylene oxide polymer comprises about 75 to 99% w/w of the core of the tablet. In one embodiment, the polyethylene oxide polymer comprises about 80 to 99% w/w of the core of the tablet. In one embodiment, the polyethylene oxide polymer comprises about 85 to 99% w/w of the core of the tablet. In one embodiment, the polyethylene oxide polymer comprises about 85 to 98% w/w of the core of the tablet.

In one embodiment, the 6-TG and polyethylene oxide polymer are in a ratio of about 1:20 to 1:50, preferably about 1:30 to 1:40, more preferably about 1:35.

In one embodiment, the 6-TG and polyethylene oxide polymer having a molecular weight of about 2,000,000 g/mol are in a ratio of about 1:30 to 1:40, preferably about 1:35.

In one embodiment, the pharmaceutical composition comprises a lubricant.

In one embodiment, the core of the pharmaceutical composition comprises a lubricant.

In one embodiment, the lubricant is selected from any one or more of magnesium stearate, stearic acid, calcium stearate, zinc stearate, polyethylene glycol, talc, sodium lauryl sulphate, magnesium lauryl sulphate, adipic acid, and paraffin.

In one embodiment, the lubricant comprises magnesium stearate.

In one embodiment, the pharmaceutical composition comprises about 0.2 to 5% w/w of the lubricant.

In one embodiment, the core of the pharmaceutical composition comprises about 0.2 to 5% w/w of the lubricant.

In one embodiment, the core of the pharmaceutical composition comprises about 0.5 to 2% w/w of the lubricant.

In one embodiment, wherein the pharmaceutical composition is a tablet, the tablet comprises a coating.

In one embodiment, the coating is an enteric coating.

In one embodiment, the enteric coating is capable of dissolving at a pH of at least 5.5, about 5.5 or greater than 5.5. In another embodiment, the enteric coating is capable of dissolving at a pH of at least 6.5, about 6.5 or greater than 6.5.

In a preferred embodiment, the enteric coating comprises a polymer capable of dissolving at a pH of at least 5.5, about 5.5, above about 5.5, at least 6.5, about 6.5, or above about 6.5. In one embodiment, the polymer of the enteric coating comprises cellulose acetate phthalate, hydroxy propyl methyl cellulose acetate succinate, a methacrylic acid copolymer, or combinations thereof. In one embodiment, the methacrylic acid copolymer comprises poly(methyl acrylate-co-methyl methacrylate-co-methacrylic acid), methacrylic acid-ethyl acrylate 1:1 copolymer, methacrylic acid-methyl methacrylate 1:1 copolymer, methacrylic acid-methyl methacrylate 1:2 copolymer, or mixtures thereof.

In one embodiment, the methacrylic acid-ethyl acrylate (1:1) copolymer has a molecular weight of between about 200,000 g/mol and about 400,000 g/mol.

In one embodiment, the enteric coating comprises one or more excipients selected from: a plasticiser, an anti-tacking agent, a surfactant, an antifoam agent, and mixtures thereof.

In one embodiment, the enteric coating comprises about 5 to 20% w/w of the tablet. In one embodiment, the enteric coating comprises about 5 to 15% w/w of the tablet. In one embodiment, the enteric coating comprises about 7 to 12% w/w of the tablet. In one embodiment, the enteric coating comprises about 10% w/w of the tablet.

In one embodiment, the pharmaceutical composition is a tablet containing 10 mg of 6-TG which is formulated to release 6-TG at a rate of from about 0.2 to 0.8 mg/hour for at least 12 hours when the release is measured at a pH of about 6.8 when subject to an in vitro dissolution test as described herein.

In one embodiment, the pharmaceutical composition is a tablet containing 10 mg of 6-TG which is formulated to release 6-TG at a rate of from about 0.3 to 0.5 mg/hour for at least 20 hours when the release is measured at a pH of about 6.8 when subject to an in vitro dissolution test as described herein.

In one embodiment, the pharmaceutical composition is formulated to provide approximately zero-order kinetics of 6-TG release.

In one embodiment, the pharmaceutical composition is formulated to provide approximately zero-order kinetics of 6-TG release when the release is measured at a pH of about 6.8 when subject to an in vitro dissolution test as described herein.

In one embodiment, the pharmaceutical composition is formulated to release 6-TG at an approximately constant rate with an R² value of between about 0.98 and 1 when the release is measured at a pH of about 6.8 when subject to an in vitro dissolution test as described herein.

In one embodiment, the core provides release of at least about 30% of the 6-TG in vitro at about pH 5.5 to about pH 7.5 (preferably pH 6.8) by approximately 10 hours.

In one embodiment, the pharmaceutical composition provides release of at least 30% of the 6-TG in vitro at about pH 5.5 to about pH 7.5 (preferably pH 6.8) by approximately 10 hours.

In a preferred embodiment, the pharmaceutical composition provides release of at least 70% of the 6-TG in vitro at about pH 5.5 to about pH 7.5 (preferably pH 6.8) by approximately 20 hours.

In another preferred embodiment, the pharmaceutical composition provides release of at least 85% of the 6-TG in vitro at about pH 5.5 to about pH 7.5 (preferably pH 6.8) by approximately 30 hours, preferably at least 90%, more preferably at least 95%.

In one embodiment, the core provides release of less than about 10% of the 6-TG in vitro at less than about pH 5.5 (preferably pH 1.1) by approximately 2 hours.

In one embodiment, the pharmaceutical composition provides release of less than about 10% of the 6-TG in vitro at less than about pH 5.5 (preferably pH 1.1) by approximately 2 hours.

In one embodiment, the pharmaceutical composition provides release of less than 80% of the 6-TG in vitro at about pH 5.5 to about pH 7.5 (preferably pH 6.8) by approximately 10 hours.

In one embodiment, the pharmaceutical composition provides release of less than 60% of the 6-TG in vitro at about pH 5.5 to about pH 7.5 (preferably pH 6.8) by approximately 10 hours.

In one embodiment, the pharmaceutical composition provides release of less than 50% of the 6-TG in vitro at about pH 5.5 to about pH 7.5 (preferably pH 6.8) by approximately 10 hours.

In one embodiment, the pharmaceutical composition provides release of: at least 30% of the 6-TG in vitro at about pH 5.5 to about pH 7.5 (preferably pH 6.8) by approximately 10 hours, and less than 80% of the 6-TG in vitro at about pH 5.5 to about pH 7.5 (preferably pH 6.8) by approximately 10 hours, preferably less than 60%, more preferably 50%.

In one embodiment, the pharmaceutical composition provides release of: at least 70% of the 6-TG in vitro at about pH 5.5 to about pH 7.5 (preferably pH 6.8) by approximately 20 hours, and less than 80% of the 6-TG in vitro at about pH 5.5 to about pH 7.5 (preferably pH 6.8) by approximately 10 hours, preferably less than 60%, more preferably 50%.

In one embodiment, the pharmaceutical composition provides release of:
at least 85% of the 6-TG in vitro at about pH 5.5 to about pH 7.5 (preferably pH 6.8) by approximately 30 hours, preferably at least 90%, more preferably at least 95%, and
less than 80% of the 6-TG in vitro at about pH 5.5 to about pH 7.5 (preferably pH 6.8) by approximately 10 hours, preferably less than 60%, more preferably 50%.

In one embodiment, the pharmaceutical composition provides release of:
at least 30% of the 6-TG in vitro at about pH 5.5 to about pH 7.5 (preferably pH 6.8) by approximately 10 hours,
at least 70% of the 6-TG in vitro at about pH 5.5 to about pH 7.5 (preferably pH 6.8) by approximately 20 hours,
at least 85% of the 6-TG in vitro at about pH 5.5 to about pH 7.5 (preferably pH 6.8) by approximately 30 hours, preferably at least 90%, more preferably at least 95%, and
less than 80% of the 6-TG in vitro at about pH 5.5 to about pH 7.5 (preferably pH 6.8) by approximately 10 hours, preferably less than 60%, more preferably less than 50%.

In one embodiment, the pharmaceutical composition provides release of at least 80% of the 6-TG in the distal intestine.

In one embodiment, the pharmaceutical composition provides release of at least 80% of the 6-TG by approximately 32 hours from oral administration, preferably at least 90%, more preferably at least 95%.

In one embodiment, the pharmaceutical composition provides release of at least 30% of the 6-TG in vivo about 12 hours after oral administration.

In one embodiment, the pharmaceutical composition is formulated to minimise substantial systemic concentrations of 6-TG.

In one embodiment, the pharmaceutical composition comprises an additional active agent for treating a disease or condition responsive to 6-TG.

In another aspect, the present invention provides a pharmaceutical composition as described herein wherein the composition is for treating an inflammatory disease or condition of the distal intestine responsive to 6-TG, for example inflammatory bowel disease.

In another aspect, the present invention provides use of a pharmaceutical composition as described herein wherein the composition is for treating an inflammatory disease or condition of the distal intestine responsive to 6-TG, for example inflammatory bowel disease.

In a further aspect, the present invention provides a method for treating a disease or condition of the distal intestine that responds to 6-TG in an individual in need thereof, the method comprising administering a pharmaceutical composition as described herein, wherein the 6-TG is released in the distal intestine (as defined herein). Preferably, substantial systemic concentrations of 6-TG are minimised. Preferably, the 6-TG is metabolized by luminal bacterial or diseased mucosa at a site of inflammation associated with the disease or condition.

In a yet further aspect, the present invention provides a method as described herein further comprising administering an additional active agent for treating a disease or condition responsive to 6-TG.

In yet another aspect, the present invention provides the use of 6-TG, a polyethylene oxide polymer having a molecular weight of between about 900,000 and about 9,000,000 g/mol, optionally including an enteric coating, in the manufacture of a medicament, wherein the medicament is formulated to treat a disease or condition of the distal intestine that responds to 6-TG by releasing the 6-TG in the distal intestine (as defined herein).

In one embodiment, the medicament or the pharmaceutical composition is formulated to provide a dose of about 1 to 50 mg 6-TG per day.

In one embodiment, the medicament or the pharmaceutical composition is formulated to provide a dose of about 1 to 40 mg 6-TG per day.

In one embodiment, the medicament or the pharmaceutical composition is formulated to provide a dose of about 1 to 30 mg 6-TG per day.

In one embodiment, the medicament or the pharmaceutical composition is formulated to provide a dose of about 5 to 30 mg 6-TG per day.

In one embodiment, the medicament or the pharmaceutical composition is formulated to provide a dose of about 5 to 20 mg 6-TG per day.

In one embodiment, the medicament or the pharmaceutical composition is formulated to provide a dose of about 10 to 20 mg 6-TG per day.

In one embodiment, the medicament or the pharmaceutical composition is formulated to provide a dose of about 10 mg 6-TG per day.

For the avoidance of doubt, the embodiments may apply alone or in any combination of two or more thereof to any one or more of the aspects set forth above where the context allows.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1 is a schematic diagram illustrating the metabolic pathway of the thiopurines. Thiopurines: AZA: azathiopurine; 6MP: 6-mercaptopurine; TG: 6-thioguanine; Thiopurine nucleotides: TGMP: thioguanosine monophosphate; TGDP: thioguanosine diphosphate; TGTP: thioguanosine triphosphate; Anabolic enzymes & metabolites: IMPDH: inosine monophosphate dehydrogenase; GMPS: guanosine monophosphate synthase; TIMP: thioinosine monophosphate; ITPA: inosine triphosphate; HPRT: hypoxanthine guanine phosphoriboxy transferase.

FIG. 2 shows a graphical representation of the cumulative release of 6-TG from core Formulation 1 over 30 hours.

FIG. 3 shows a graphical representation of the cumulative release of 6-TG from core Formulation 2 over 30 hours.

FIG. 4 shows a graphical representation of the cumulative release of 6-TG from core Formulation 3 over 30 hours.

FIG. 5 shows a graphical representation of the cumulative release of 6-TG from core Formulation 4 over 30 hours.

FIG. 6 shows a graphical representation of the cumulative release of 6-TG from Formulation 5 (enteric coated) over 36 hours (hours 1-2 in pH 1.1, hour 3 in pH 6.5, hours 4-36 in pH 6.8).

FIG. 7A and B show graphical representations of a typical thioguanine standard UV spectrum (A) and a typical sample solution UV spectrum (B).

FIG. 8 shows a graphical representation of the cumulative release of 6-TG from the Formulation of Example 10 (enteric coated) over 30 hours (hours 1-2 in pH 1.1, hour 3 in pH 5.5, hours 4-30 in pH 6.8).

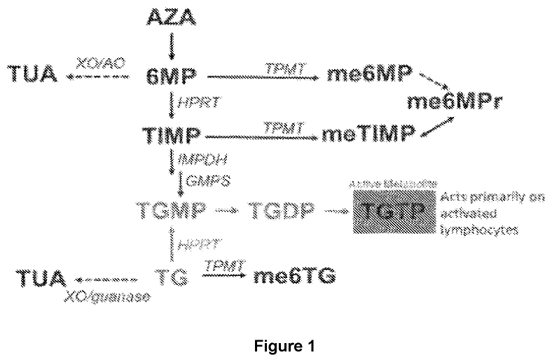

### DEFINITION OF TERMS

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

As used herein, the term “about” refers to a quantity, level, concentration, value, size, or amount that varies by as much as 10% or even as much as 9%, 8%, 7%, 6%, 5%, 4%, 3%, 2%, 1%, to a reference quantity, level, concentration, value, size, or amount.

As used herein, except where the context requires otherwise, the term “comprise” and variations of the term, such as “comprising”, “comprises” and “comprised”, are not intended to exclude further additives, components, integers or steps.

As used herein the term “distal intestine” refers to the section of intestine comprising the distal ileum and large intestine. In some embodiments the relevant section of the distal intestine is the large intestine.

As used herein, the term “large intestine” refers to the colon and rectum. The term “colon” refers to the section of intestine consisting of the caecum, ascending colon, transverse colon, descending colon and sigmoid colon. In some embodiments the relevant section of the large intestine is the colon. In some embodiments the relevant section of the large intestine is the rectum.

As used herein, the term “proximal and mid small intestine” refers to the duodenum, jejunum and the portion of the ileum that excludes the distal ileum (approximately the last less than or equal to 100 cm of the ileum).

As used herein, the phrase “diseases or conditions of the distal intestine that respond to 6-TG”, “diseases or conditions of the distal ileum and/or large intestine that respond to 6-TG”, “diseases or conditions of the distal ileum, colon and/or rectum that respond to 6-TG”, and similar refers to any disease or condition of the distal ileum, colon or rectum which can be treated by 6-TG, including treatment of any symptom of the disease or condition. Preferably the disease or condition is an inflammatory disease or condition. Examples of such inflammatory diseases or conditions include ulcerative colitis, Crohn's disease and IBD-U (IBD-Undifferentiated).

When used herein, the phrase “inflammatory bowel disease” is a condition that usually affects the distal intestine and includes ulcerative colitis, Crohn's disease and IBD-U (IBD-Undifferentiated).

The term “extended-release formulation” refers to a formulation that is converted or degraded or metabolised into, or provides a source of, the active component that it contains (e.g., 6-TG) over an extended period of time. Such formulations may also be referred to in the art as slow release, or sustained release formulations.

The term “extended release” refers to release of the active pharmaceutical ingredient (6-TG) from a pharmaceutical formulation over an extended period of time, thus providing continuous local delivery of 6-TG as the formulation passes through the distal intestine. In some embodiments the extended release of 6-TG has substantially zero order kinetics.

The terms “individual”, “patient” and “subject” are used interchangeably herein to refer to individuals of human or other animal origin and includes any individual it is desired to examine or treat using the methods of the invention. However, it will be understood that these terms do not imply that symptoms are present. Suitable animals that fall within the scope of the invention include, but are not restricted to, humans, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes, birds, reptiles).

The phrase “6-TG side-effects” and similar expressions refers to the adverse or undesirable side-effects caused by 6-TG, and/or a 6-TG metabolite thereof, including 6-TGTP (and/or other metabolites), including but not limited to vascular hepatotoxicity, including sinusoidal obstructive syndrome (SOS), veno-occlusive disease (VOD) and nodular regenerative hyperplasia (NRH); and excessive immunosuppression or myelosuppression; and alopecia.

By “pharmaceutically acceptable excipient, carrier or diluent” is meant a solid or liquid filler, diluent or encapsulating substance that can be safely used in topical or systemic administration.

The terms “treat”, “treating” or “treatment” as used herein cover the treatment of a disease or condition responsive to 6-TG in an individual having the disease or condition, and includes: inhibiting the disease or condition, i.e., arresting its development; relieving the disease or condition, i.e., causing regression of the disease or condition; or relieving the symptoms resulting from the disease or condition, i.e., relieving pain or inflammation without addressing the underlying disease or condition.

It will be understood that the invention disclosed and defined in this specification extends to all alternative combinations of two or more of the individual features mentioned or evident from the text or drawings. All of these different combinations constitute various alternative aspects of the invention.

### DESCRIPTION OF THE INVENTION

The present invention relates to a pharmaceutical composition comprising 6-TG, and a polyethylene oxide polymer having a molecular weight of between about 900,000 g/mol and about 9,000,000 g/mol. The composition may be formulated for release of 6-TG in the distal intestine, preferably the large intestine. Suitably, the composition is formulated to release 6-TG substantially in the distal intestine. Suitably, the composition is formulated for oral administration. Suitably the pharmaceutical composition is formulated to provide a delayed release to release 6-TG substantially in the distal intestine over an extended period following oral administration.

Suitably, the composition is formulated for extended release of 6-TG in the distal intestine. Suitably, the composition releases at least 80% of the 6-TG in the distal intestine. In some embodiments, the section of the distal intestine is the large intestine. Suitably, the composition is for treating a disease or condition of the distal intestine responsive to 6-TG. Suitably, the composition is for treating IBD, e.g. ulcerative colitis, Crohn's disease or IBD-U. In some embodiments, the compositions may be used for treating microscopic colitides; e.g., lymphocytic (entero) colitis and collagenous (entero) colitis.

The polyethylene oxide polymer has a molecular weight of between about 900,000 g/mol and about 9,000,000 g/mol. Preferably, the polyethylene oxide polymer has a molecular weight of greater than about 900,000 g/mol and less than about 9,000,000 g/mol.

Preferably, the polyethylene oxide polymer has a molecular weight of between about 1,000,000 g/mol and about 6,000,000 g/mol; between about 1,000,000 g/mol and about 5,000,000 g/mol; between about 1,000,000 g/mol and about 4,000,000 g/mol; between about 1,000,000 g/mol and about 3,000,000 g/mol. More preferably, the polyethylene oxide polymer has a molecular weight of greater than about 1,000,000 g/mol and less than about 6,000,000 g/mol; greater than about 1,000,000 g/mol and less than about 5,000,000 g/mol; greater than about 1,000,000 g/mol and less than about 4,000,000 g/mol; greater than about 1,000,000 g/mol and less than about 3,000,000 g/mol.

Preferably, the polyethylene oxide polymer has a molecular weight of about 900,000 g/mol; 1,000,000 g/mol; 1,100,000 g/mol; 1,200,000 g/mol; 1,300,000 g/mol; 1,400,000 g/mol; 1,500,000 g/mol; 1,600,000 g/mol; 1,700,000 g/mol; 1,800,000 g/mol; 1,900,000 g/mol; 2,000,000 g/mol; 2,000,000 g/mol; 2,100,000 g/mol; 2,200,000 g/mol; 2,300,000 g/mol; 2,400,000 g/mol; 2,500,000 g/mol; 2,600,000 g/mol; 2,700,000 g/mol; 2,800,000 g/mol; 2,900,000 g/mol; 3,000,000 g/mol; 3,100,000 g/mol; 3,200,000 g/mol; 3,300,000 g/mol; 3,400,000 g/mol; 3,500,000 g/mol; 3,600,000 g/mol; 3,700,000 g/mol; 3,800,000 g/mol; 3,900,000 g/mol; 4,000,000 g/mol; 4,100,000 g/mol; 4,200,000 g/mol; 4,300,000 g/mol; 4,400,000 g/mol; 4,500,000 g/mol; 4,600,000 g/mol; 4,700,000 g/mol; 4,800,000 g/mol; 4,900,000 g/mol; 5,000,000 g/mol; 5,100,000 g/mol; 5,200,000 g/mol; 5,300,000 g/mol; 5,400,000 g/mol; 5,500,000 g/mol; 5,600,000 g/mol; 5,700,000 g/mol; 5,800,000 g/mol; 5,900,000 g/mol; 6,000,000 g/mol; 6,100,000 g/mol; 6,200,000 g/mol; 6,300,000 g/mol; 6,400,000 g/mol; 6,500,000 g/mol; 6,600,000 g/mol; 6,700,000 g/mol; 6,800,000 g/mol; 6,900,000 g/mol; 7,000,000 g/mol; 7,100,000 g/mol; 7,200,000 g/mol; 7,300,000 g/mol; 7,400,000 g/mol; 7,500,000 g/mol; 7,600,000 g/mol; 7,700,000 g/mol; 7,800,000 g/mol; 7,900,000 g/mol; 8,000,000 g/mol; 8,100,000 g/mol; 8,200,000 g/mol; 8,300,000 g/mol; 8,400,000 g/mol; 8,500,000 g/mol; 8,600,000 g/mol; 8,700,000 g/mol; 8,800,000 g/mol; 8,900,000 g/mol; or 9,000,000 g/mol.

Preferably, the polyethylene oxide polymer has a molecular weight of greater than about 1,000,000 g/mol and less than about 6,000,000 g/mol. This molecular weight range has been shown to provide extended release of 6-TG over about 30 hours in vitro at pH 6.8, with a substantially linear release over the majority of the release time.

Most preferably, the polyethylene oxide polymer has a molecular weight of about 2,000,000 g/mol.

The polyethylene oxide polymer may comprise one or more polymers. In one embodiment, the polyethylene oxide polymer comprises substantially one polymer of a single approximate molecular weight. For example, a polyethylene oxide polymer having a molecular weight of between about 900,000 g/mol and about 9,000,000 g/mol may comprise substantially one polymer of a single approximate molecular weight of for example about 2,000,000 g/mol. In another embodiment, the polyethylene oxide polymer comprises two or more polymers of different approximate molecular weights, wherein each polymer has a molecular weight within the defined molecular weight range. For example, the polyethylene oxide polymer having a molecular weight of between about 900,000 g/mol and about 9,000,000 g/mol may comprise two or more polyethylene oxide polymers wherein, for example, a first polyethylene oxide polymer has a molecular weight of about 2,000,000 g/mol, and a second polyethylene oxide polymer has a molecular weight of about 7,000,000 g/mol. The composition may also include, in addition to the polyethylene oxide polymer having a molecular weight of between about 900,000 g/mol and about 9,000,000 g/mol, a further polyethylene oxide polymer with higher or lower molecular weight.

As will be apparent to those skilled in the art, the molecular weights of polyethylene oxide polymer are approximate due the nature of polymerisation reactions. Polyethylene oxide polymer is commercially available in a number of different weights. The weights are the approximate or average weight. For example, POLYOX™ (polyethylene oxide) WSR N-60K (PEO 2,000,000) is available from Dupont, with a viscosity range 2000-4000 mPa-sec (cP) for 2% weight percentage of the aqueous solution measured at 25° C. on a Brookfield Viscometer, Model RVF, Spindle No./Speed, rpm 3/10.

In some embodiments, the pharmaceutical composition is a tablet. The tablet may comprise a core and a coating. An exemplary core comprises 6-TG and a polyethylene oxide polymer having a molecular weight between about 900,000 g/mol and about 9,000,000 g/mol, forming a drug-polymer matrix. An exemplary tablet comprises the core and an enteric coating.

In some embodiments, the tablet comprises about 1 to 50 mg, 1 to 40 mg, 1 to 30 mg, 1 to 20 mg, or 5 to 20 mg of 6-TG. Preferably, the tablet comprises about 1 mg, 2 mg, 3 mg, 4 mg, 5 mg, 10 mg, 20 mg, 25 mg, 30 mg, 40 mg or 50 mg of 6-TG. Preferably the tablet comprises about 10 mg of 6-TG.

In some embodiments, the tablet weighs about 300 to 500 mg, or about 300 mg to 450 mg. Preferably, the tablet weights about 350 mg to 400 mg. Preferably, the core component of the tablet weighs about 100 mg, 200 mg, 300 mg, 400 mg, or 500 mg. More preferably, the core component of the tablet weighs about 300 mg to 400 mg, or about 320 mg to about 380 mg. Even more preferably the core weights about 360 mg.

In some embodiments, 6-TG is present in an amount of about 1 to 30% w/w of the core, or about 1 to 10% w/w of the core, including about 1%, 2%, 3%, 4%, 5%, 6%, 7%, 8%, 9% and 10% w/w of the core, preferably about 2 to about 3% w/w.

In some embodiments 6-TG is present in an amount of about 1 to 30% w/w of the tablet, or about 1 to 20% w/w, or about 1 to 10% w/w or about 1 to 5% w/w or about 2 to 4% w/w.

In some embodiments the polyethylene oxide polymer having a molecular weight of between about 900,000 g/mol and about 9,000,000 g/mol is present in an amount of about 70 to about 99% w/w of the core, preferably about 75 to about 99% w/w, 80 to 99% w/w, 85 to 99% w/w, 85 to 98% w/w, especially about 90 to about 98% w/w of the core, including about 90%, 91%, 92%, 93%, 94%, 95%, 96%, 97%, and 98% w/w of the core, most preferably about 97% w/w.

In some embodiments the tablet also comprises one or more pharmaceutically acceptable excipients such as a carrier, diluent, binder and/or lubricant, including those that impart the desired consistency, strength, viscosity, texture and appearance.

Ethyl cellulose may be used to modify the release rate of the core (for example to extend the release time). However, preferably, the pharmaceutical composition comprising an enteric coating does not contain ethyl cellulose. The use of ethyl cellulose may also adversely affect the release kinetics.

Suitable binders include disaccharides such as lactose; polysaccharides such as starches for example, corn starch, starch paste; and sugar alcohols such as mannitol.

Suitable lubricants include fats, such as magnesium stearate; talc; or silica, for example, fumed silica. In one embodiment, the lubricant is selected from any one or more of magnesium stearate, stearic acid, calcium stearate, zinc stearate, polyethylene glycol, talc, sodium lauryl sulphate, magnesium lauryl sulphate, adipic acid, and paraffin, preferably magnesium stearate.

The lubricant may be present in an amount of about 0.2 to 5% w/w of the core, especially about 0.2 to 4% w/w of the core, especially about 0.5 to 2% w/w of the core. In one embodiment, the lubricant is present in an amount of about 1 to 2.5% w/w of the core. The lubricant may be present in an amount of about 0.2 to 5% w/w of the pharmaceutical composition.

The pharmaceutical composition may comprise about 1 to 30% w/w of the 6-TG, or about 1 to 20% w/w of the 6-TG, or about 1 to 10% w/w of the 6-TG, or about 1 to 5% w/w of the 6-TG or about 2 to 4% w/w of the 6-TG.

As will be apparent to those skilled in the art, the 6-TG may be available and/or used as a hydrate (for example heptahydrate). When considering the percentage or ratio of the 6-TG in the pharmaceutical composition the mass of the water in a hydrate should not be included.

The ratio of compound of 6-TG to polyethylene oxide polymer may be about 1:20 to 1:50, for example about 1:30 to 1:40; about 1:35.

In some embodiments the ratio of 6-TG to polyethylene oxide polymer having a molecular weight of about 2,000,000 g/mol is about 1:30 to 1:40, preferably about 1:35.

Where the pharmaceutical composition is a tablet comprising a core and a coating, the coating is preferably an enteric coating.

The present disclosure may relate to a pharmaceutical composition comprising an oral formulation of 6-TG, a polyethylene oxide polymer having a molecular weight of between about 900,000 g/mol and 9,000,000 g/mol, and an enteric coating. The composition may be formulated for release of 6-TG in the distal intestine, preferably the large intestine.

An enteric coating controls or delays the release of 6-TG by reducing release in the stomach and/or by reducing or substantially preventing release of 6-TG prior to the tablet reaching the distal intestine. Suitable enteric coatings are capable of dissolving at a pH of at least 5.5, about 5.5, or greater than 5.5.

In a preferred embodiment, the enteric coating includes a polymer capable of dissolving at a pH of at least 5.5, about 5.5, or greater than 5.5. Preferably, the polymer dissolves at a pH from about 5.5 to about 8. Advantageously the enteric coating comprising a polymer capable of dissolving at a pH of greater than about 5.5 minimises or at least reduces disintegration of the composition in the stomach, proximal and mid small intestine.

Preferably, the polymer of the enteric coating capable of dissolving at a pH of at least 5.5 comprises cellulose acetate phthalate, hydroxy propyl methyl cellulose acetate succinate, and/or a co-polymers of acrylic acids and their esters or methacrylic acids or their esters. Preferably the polymer of the enteric coating comprises a methacrylic acid copolymer. Preferably, the polymer comprises poly(methyl acrylate-co-methyl methacrylate-co-methacrylic acid), methacrylic acid-ethyl acrylate [1:1] copolymer, methacrylic acid-methyl methacrylate 1:1 copolymer, methacrylic acid-methyl methacrylate 1:2 copolymer, or mixtures thereof. More preferably, the methacrylic acid-ethyl acrylate (1:1) copolymer has a molecular weight of between about 200,000 g/mol and about 400,000 g/mol, preferably about 320,000 g/mol. In one embodiment the coating comprises Eudragit® L30 D-55 or Acryl-EZE powder dispersion, preferable Acryl-EZE 93F19255.

An enteric coating may be applied to a tablet using conventional techniques known in the art. For example, a coating may be applied using spray coating, dip coating, fluid bed or pan coating technology as described in, for example, Remington: The Science and Practice of Pharmacy, Loyd V. Allen, Jr (Ed), The Pharmaceutical Press, London, 22nd Edition, September 2012 at Chapter 46.

In one embodiment, the enteric coating comprises one or more pharmaceutically acceptable excipients, carriers and/or diluents. Such excipients, diluents and carriers enable the compositions to be formulated into/onto dosage forms such as tablets (including pills, capsules, and the like), for oral administration to an individual. Acceptable excipients, diluents and carriers are well known to those skilled in the art and include, but are not restricted to, plasticisers, anti-tacking agents, surfactants, antifoam agents, pigments and mixtures thereof. Suitable plasticisers include, but are not limited to, triethyl citrate, polyethylene glycol, 1,2-propylene glycol, triactin, dibutyl phthalate, tributyl citrate, acetyltriethyl citrate and acetyltributyl citrate. Suitable anti-tacking agents include glycerol monostearate, talc, silica, calcium silicate. Suitable surfactants include polysorbate 80. Suitable antifoam agents include simethicone, dimethicone, oleyl alcohol.

In a preferred embodiment, the enteric coating comprises excipients selected from: glycerol monostearate, triethyl citrate and polysorbate 80, talc, silica, polyethylene glycol, triethyl citrate, simethicone and combinations thereof. In a particularly preferred embodiment, the coating includes talc, silica, polyethylene glycol, and triethyl citrate. In yet a further embodiment, the enteric coating comprises a plasticiser and an antifoam agent.

### Release Profile

Preferably, the pharmaceutical composition comprising an enteric coating provides a delayed release following oral administration and/or at pH less than about 5.5 in vitro.

Preferably, the pharmaceutical composition comprising an enteric coating minimises release of 6-TG at a pH of less than about 5.5; 5.4; 5.3; 5.2; 5.1; 5.0; 4.9; 4.8; 4.7; 4.6; 4.5; 4.4; 4.3; 4.2; 4.0; 3.9; 3.8; 3.7; 3.6; 3.5; 3.4; 3.3; 3.2; 3.1; 3.0; 2.9; 2.8; 2.7; 2.6; 2.5; 2.4; 2.3; 2.2; 2.1; 2.0; 1.9; 1.8; 1.7; 1.6; 1.5.

Preferably, the tablet comprising an enteric coating provides release of no more than about 10% of the 6-TG in vitro at a pH less than about 5.5 (preferably about pH 1.1) for a period of about 2 hours or less when subject to an in vitro dissolution test as described herein, preferably no more than about 9%, 8%, 7%, 6%, 5%, 4%, 3%, 2%, or 1%.

Preferably, the pharmaceutical composition formulated for oral administration comprising an enteric coating provides release of no more than about 10% of the 6-TG by approximately 2 hours from oral administration, or no more than about 8% of the 6-TG by approximately 2 hours from oral administration, or no more than about 5% of the 6-TG by approximately 2 hours from oral administration.

Preferably, the pharmaceutical composition comprising an enteric coating is formulated to release 6-TG at a pH of from about 5.5 and above, for example about pH 6.8, 7, 7.5, or 8 in vivo or in vitro.

Preferably, the pharmaceutical composition provides release of at least 30% of the 6-TG in vitro at about pH 5.5; 6; 6.5; 6.8; 7; 7.5; most preferably at about pH 6.8, by approximately 10 hours. Preferably the pharmaceutical composition provides release of at least 30%, of the 6-TG in vitro at about pH 5.5 to about pH 7.5, most preferably at about pH 6.8, by approximately 10 hours.

The pharmaceutical composition preferably provides release of at least 70% of the 6-TG in vitro at about pH 5.5 to about pH 7.5 (preferably pH 6.8) by approximately 20 hours. Preferably, the pharmaceutical composition provides release of at least 70%, at least 71%, at least 72%, at least 73%, at least 74%, at least 75%, at least 76% at least 77%, at least 78%, at least 79%, at least 80%, at least 81%, at least 82%, at least 83%, or at least 84% of the 6-TG in vitro at about pH 5.5 to about pH 7.5, most preferably at about pH 6.8, by approximately about 20 hours.

Preferably the pharmaceutical composition provides release of at least 85%, at least 90%, at least 91%, at least 92%, at least 93%, at least 94%, at least 95%, at least 96%, at least 97%, at least 98%, or at least 99% of the 6-TG in vitro at about pH 5.5 to about pH 7.5, most preferably at about pH 6.8, by approximately 30 hours, more preferably at least 90%, even more preferably at least 95%.

In some instances, the composition is defined herein to provide release of at least a certain percentage of 6-TG either in vitro, or in vivo, by a specified period of time. As will be apparent to the skilled person, this means that at least, at a minimum, the defined percentage of 6-TG has already been released at the specified time point. For example, at least 35% of the 6-TG has already been released by 10 hours. More than 35% of the 6-TG may have been released by 10 hours, for example 45% or 50%, of the 6-TG may have been released by 10 hours. This also means that no more than 65% of the 6-TG is still remaining, yet to be released, after 10 hours, for example no more than 55% or 50% may be remaining, yet to be released after 10 hours.

Preferably the pharmaceutical composition provides release of less than about 10% of the 6-TG in vitro at less than about pH 5.5 (preferably pH 1.1) by approximately 2 hours.

Preferably the pharmaceutical composition provides release of less than 80% of the 6-TG in vitro at about pH 5.5 to about pH 7.5 (preferably pH 6.8) by approximately 10 hours, or of less than 60% of the 6-TG, or less than 50% of the 6-TG.

Methods to determine the in vitro release profile to mimic pH changes in the gastrointestinal tract will be known by those in the art. An exemplary method to determine the in vitro release profile includes testing using dissolution apparatus appropriate for tablets using media containing a suitable buffer adjusted to the relevant pH. The dissolution test can utilise an initial pH which is adjusted during the course of the test. In vitro dissolution testing as described herein is performed in dissolution baskets in buffer at required pH (pH 6.8 unless otherwise stated). The dissolution is calculated by measuring the US-vis absorbance of the sample at 341 nm compared to a solution of known concentration of thioguanine in the same buffer.

In a preferred embodiment, there is provided a pharmaceutical composition comprising 6-thioguanine (6-TG), and a polyethylene oxide polymer having a molecular weight of greater than about 900,000 g/mol and less than about 7,000,000 g/mol, formulated for oral administration comprising an enteric coating, wherein the composition provides release of:
no more than about 10% of the 6-TG in vitro at a pH of less than about 5.5, preferably about pH 1.1, for a period of approximately 2 hours, preferably no more than about 9%, 8%, 7%, 6%, 5%, 4%, 3%, 2%, or 1%,
no more than about 20-30% of the 6-TG in vitro at about pH 5.5 to about pH 7.5, preferably about pH 6.8, for a period of approximately 6 hours or less, preferably no more than about 20%, 21%, 22%, 23%, 24%, 25%, 26%, 27%, 28%, 29% or 30%, and
release of at least 85% of the 6-TG in vitro at a pH of about 5.5 to about 7.5, preferably pH 6.8, by approximately 30 hours or more.

As will be apparent to those skilled in the art, an in vivo release profile may be approximated using in vitro methods.

The transit time of an oral composition from mouth to anus will vary considerably from individual to individual, and many parameters such as age, solid or liquid food intake, diet, weight, sex, health, disease state of gut, etc., will impinge on the actual transit time. Not all ingested material transits at the same rate, so that typically transit is measured as 50% clearance of oral markers that are ingested at the one time. The transit time from mouth to entry into duodenum typically varies from approximately 30 minutes to 90 minutes. The average time for transit through the small intestine from duodenum to entry into caecum is approximately 2-6 hours. The typical transit time from mouth to caecum is considered to be approximately 4-8 hours. The transit time through the colon (from caecum to anus) is approximately 12 to 36 hours. The typical transit time from mouth to anus is thus considered to be approximately 36 hours, although this may vary widely. Typical transit times may vary from about 16 hours to about 48 hours.

Suitably, the delayed-released and extended-release enteric coated oral formulations of the present invention are formulated so as to release or provide 6-TG to the distal intestine, preferably the large intestine. The enteric coating provides the delay in release by limiting release below about pH 5.5 to minimise or reduce release in the stomach.

In some embodiments the oral formulation provides a 6-TG release of at least 35% of the total 6-TG in the distal intestine, preferably the large intestine. In some embodiments the oral formulation provides a 6-TG release of at least 35%, 40%, 45%, 50%, 55%, 60%, 65%, 70%, 75%, 80%, 85%, 90%, 91%, 92%, 93%, 94%, 95%, 96%, 97%, 98%, or 99% of the total 6-TG in the distal intestine, preferably the large intestine. In some embodiments the oral formulation provides a 6-TG release of at least 80% of the total 6-TG in the distal intestine, and preferably in the large intestine.

Preferably, compositions comprising a polyethylene oxide polymer having a molecular weight of about 2,000,000 g/mol provides release of at least 80%, at least 85%, at least 90%, at least 91%, at least 92%, at least 93%, at least 94%, at least 95%, at least 96%, at least 97%, at least 98%, or at least 99% of the 6-TG by approximately 30 hours from oral administration, most preferably at least 80%. Preferably, compositions comprising a polyethylene oxide polymer having a molecular weight of about 2,000,000 g/mol provides release of at least 90%, at least 91%, at least 92%, at least 93%, at least 94%, at least 95%, at least 96%, at least 97%, at least 98%, or at least 99% of the 6-TG by approximately 30 to 40 hours from oral administration, most preferably at least 95%.

Preferably, the pharmaceutical composition formulated for oral administration comprising an enteric coating provides release of no more than about 10% of the 6-TG by approximately 2 hours from oral administration, preferably no more than about 9%, 8%, 7%, 6%, 5%, 4%, 3%, 2%, 1% or 0%, and release of at least 35% of the 6-TG by approximately 12 hours after oral administration. Preferably the pharmaceutical composition provides release of at least 80%, at least 85%, at least 90%, at least 91%, at least 92%, at least 93%, at least 94%, at least 95%, at least 96%, at least 97%, at least 98%, or at least 99% of the 6-TG by approximately 30 to 40 hours from oral administration.

Preferably, the composition is formulated to minimise substantial systemic absorption of 6-TG. Systemic absorption of orally administered 6-TG primarily occurs in the proximal small intestine. The average time for transit through the small intestine from duodenum to entry into caecum is approximately 2-6 hours. The typical transit time from mouth to caecum is considered to be approximately 4-8 hours. To minimise systemic absorption of 6-TG, the pharmaceutical composition is preferably formulated to release no more than about 15-25% of the 6-TG in vitro at about pH 5.5 to about pH 7.5, preferably about pH 6.8, for a period of approximately 6 hours or less, preferably no more than about 15%, 16%, 17%, 18%, 19%, 20%, 21%, 22%, 23%, 24%, or 25%. An in vitro release profile of no more than about 20% of the 6-TG in vitro at about pH 5.5 to about pH 7.5, preferably about pH 6.8, for a period of approximately 6 hours or less approximates an in vivo release profile wherein less than about 20% of the 6-TG is released no more than 8 hours from oral administration. Such a release profile significantly reduces systemic 6-TG from small intestinal absorption.

Preferably, the pharmaceutical composition formulated for oral administration comprising an enteric coating provides release of no more than about 10% of the 6-TG in vitro at a pH of less than about 5.5 (preferably pH about 1.1) for a period of approximately 2 hours or less. Such a release profile approximates an in vivo release profile of no more than about 10% of the 6-TG by approximately 2 hours from oral administration. Most preferably, the formulation does not release significant amounts of 6-TG at pH<5.5, preferably no more than about 10% of the 6-TG, preferably no more than about 9%, 8%, 7%, 6%, 5%, 4%, 3%, 2%, or 1%.

In a preferred embodiment, there is provided a pharmaceutical composition comprising 6-thioguanine (6-TG), and a polyethylene oxide polymer having a molecular weight of between about 900,000 g/mol and about 9,000,000 g/mol, formulated for oral administration comprising an enteric coating, wherein the composition provides release of:
no more than about 10% of the 6-TG by approximately 2 hours from oral administration, preferably no more than about 9%, 8%, 7%, 6%, 5%, 4%, 3%, 2%, or 1%, and/or
release of at least 80% of the 6-TG by approximately 30 hours from oral administration.

In preferred embodiments, the formulation provides approximately zero order kinetics of 6-TG release in the distal intestine (i.e., a linear delivery with respect to the time of extended release). A zero-order release profile advantageously provides substantially even release and distribution of 6-TG as the composition transits through the distal intestine.

Suitably, the extended-release formulation provides substantially zero order kinetics of 6-TG release from approximately 2 hours, 3, hours, 4 hours, 6 hours, 7 hours, 8 hours, 9 hours, 10 hours, 11 hours, or 12 hours, after oral administration, preferably from approximately 2 hours, or from approximately 3 hours, or from about 4 hours. Suitably, the extended release formulation provides substantially zero order kinetics of 6-TG release from approximately 2 hours or 3 hours, or 4 hours after oral administration wherein the substantially zero order release is maintained for at least a further 12 hours, 13 hours, 14 hours, 15 hours, 16 hours, 17 hour, 18 hours, 19 hours, 20 hours, 21 hours, or 22 hours, preferably at least a further 22 hours. It will be appreciated that the time end point may be egress of the formulation residue from the gut. Preferably, the formulation provides approximately zero order kinetics of 6-TG release in vitro at about pH 5.5 to about pH 7.5, preferably wherein the substantially zero order kinetics release is maintained for at least about 12 hours, 13 hours, 14 hours, 15 hours, 16 hours, 17 hour, 18 hours, 19 hours, 20 hours, 21 hours, or 22 hours, preferably at least about 22 hours. Alternatively, the pharmaceutical composition is formulated to release 6-TG at an approximately constant rate with an R² value of between about 0.98 and 1 when the release is measured at a pH of about 6.8 when subject to an in vitro dissolution test as described herein, preferably the R² value is maintained for at least about 12 hours, 13 hours, 14 hours, 15 hours, 16 hours, 17 hour, 18 hours, 19 hours, 20 hours, 21 hours, or 22 hours, preferably at least about 22 hours. The zero order and/or R² value is preferably maintained for up to about 24 hours.

In one aspect, the present invention provides a pharmaceutical composition comprising 6-TG, and a polyethylene oxide polymer having a molecular weight of between about 900,000 g/mol and about 9,000,000 g/mol, comprising an enteric coating, wherein the composition is in a form suitable for oral administration and provides for the extended release of the 6-TG after a period of about 2 to 8 hours following oral administration. Preferably, the pharmaceutical composition provides for the extended release of the 6-TG over a period extending beyond 12 hours following oral administration. Preferably the release is substantially in the large intestine. Suitably, the composition provides for extended release over a period of about 8 hours to 40 hours, preferably about 12 to 40 hours, about 16 to 40 hours, about 20 to 40 hours, about 20 to 36 hours, or about 20 to 30 hours following start of release (for example in the large intestine and/or start of release being start of release over 2% per hour). Suitably the composition provides for extended release at a pH range of 5.5 to 7.5. It will be appreciated that the time end point may be egress of the formulation residue from the gut.

At least one combination of matrix and film coating provided in the description below can used to achieve the desired release profile across the different environments encountered during transit through the GI tract. Suitably, the formulation provides a release profile in the distal intestine. More suitably, the formulation provides a release profile in the large intestine.

### Methods of Treatment

Suitably the pharmaceutical compositions described herein are useful for treating an inflammatory disease or condition of the distal intestine.

In one embodiment, the method for treating an inflammatory disease or condition of the distal intestine that responds to 6-TG in an individual in need thereof, the method comprising administering a pharmaceutical composition as described herein to the individual, wherein the 6-TG is released in the distal intestine. Preferably the inflammatory disease or condition of the distal intestine is inflammatory bowel disease (IBD). Preferably the 6-TG is released in the distal intestine. Preferably the 6-TG is released in the large intestine. Examples of forms of IBD include ulcerative colitis, Crohn's disease, and IBD-U (unclassified). Suitably, the 6-TG is converted to active drug in the distal intestine by luminal bacteria or diseased mucosa. Suitably, systemic concentrations of 6-TG are minimised. Suitably, this method minimises or at least substantially reduces undesirable 6-TG side-effects, including vascular hepatotoxicity such as SOS, and myelosuppression, observed when high hepatic portal or systemic concentrations of active or related metabolites are generated.

Also provided is a method for treating a disease or condition that responds to 6-TG in an individual in need thereof, the method comprising administering to the individual an oral composition as described herein.

In preferred embodiments, the method comprises oral administration. Suitably, the method comprises administering a pharmaceutical composition comprising an enteric coating as described herein.

In some embodiments, the method further comprises administration of another active agent in addition to 6-TG for treating a disease or condition responsive to 6-TG, including but not limited to an active agent selected from the group consisting of: mesalazine (or 5-aminosalicylic acid), balsalazide, sulfasalazine, a corticosteroid, a cyclosporine compound, an anti-TNF-a compound, and combinations thereof. The additional active agent may be administered in the same composition or compositions, or in a different composition or composition to the composition(s) comprising 6-TG. The additional active agent may be administered at the same time or at a different time to the composition(s) comprising 6-TG.

The present invention indicates combinations, kits or commercial packages comprising the 6-TG composition and a composition comprising the additional active agent. Such kits or commercial packages may include instructions for use. In some cases it may be appropriate to provide a composition comprising 6-TG and the other active agent. In some embodiments the composition substantially does not comprise 6-MP or AZA. In some embodiments the composition comprises 6-TG as the sole active pharmaceutical ingredient.

The pharmaceutical compositions should also have the required shelf life in accordance with the International Conference on Harmonisation issued by the US Food and Drug Administration.

The tablet formulations can be prepared by any suitable tableting method, although direct compression when possible is particularly advantageous and economical as it involves fewer processing steps than other processes. It is also possible to prepare tablets (including granules and pellets) using any suitable technique, including wet granulation, dry granulation and roller compaction. In exemplary embodiments, wet granulation or direct compression is used. Suitably, a wet granulation. Where ingredients (for example the 6-TG and the polyethylene oxide polymer and/or other core ingredients) are blended prior to forming into a tablet care is preferably taken to ensure a substantially uniform or homogenous blend. For example, samples of the blend may be taken to test for uniformity, preferably the samples vary in amount or relative standard deviation of 6-TG by less than about 6%, or about 5%, or about 4%, or preferably about 2%. For example, see J Pharm Innov (2015) 10:76-83 and J Pharm Innov. (2015) 10:84-97 for details of standard processes used to access blend uniformity.

### Dosage Levels

In specific embodiments, the optimal therapeutic protocol, and thus the methods of the present invention comprise administration of a total daily dose of 6-TG of about 0.1 mg/kg, 0.2 mg/kg or 0.3 mg/kg body weight to about 2.5 mg/kg body weight, for example 0.1 mg/kg body weight to 2.5 mg/kg body weight. Suitably the total daily dose of 6-TG is 0.1, 0.2 or 0.3 mg/kg body weight to 2.0 mg/kg body weight; 0.1, 0.2 or 0.3 mg/kg body weight to 1.5 mg/kg body weight; or 0.1, 0.2 or 0.3 mg/kg body weight to 1 mg/kg body weight. More suitably the total daily dose of 6-TG is from 0.1, 0.3, 0.4 or 0.5 mg/kg body weight to 1.0 mg/kg body weight, and even more suitably the total daily dose of 6-TG is about 0.1, 0.3, 0.4 or 0.5 mg/kg body weight to about 0.8 mg/kg body weight. Suitably, the dose is administered as a once daily dose.

It will be appreciated by those skilled in the art that the dosages above, although recited as a daily dosage, may not be administered on a daily basis. For example, a daily dose of 6-TG of 0.1 mg/kg/bodyweight to 1 mg/kg/body weight may be administered every 2, 3, 4, 5, 6, or 7 days (or otherwise). In an illustrative embodiment, an individual (e.g., adult) of 60 kilograms receiving a daily dose of 6-TG of 1 mg/kg/body weight (i.e., a daily dose of 60 mg) may receive 60 mg/day or may instead receive 120 mg every 2 days, or 180 mg every 3 days, etc. In another illustrative embodiment, an individual (e.g. child) of 20 kilograms may receive the same daily dose of 6-TG (1 mg/kg/body weight) equating to 20 mg/day, or 40 mg/2 days or 60 mg/3 days, etc. In another illustrative embodiment an individual (e.g., adult) of 60 kilograms receiving a daily dose of 6-TG of 0.2 mg/kg/body weight (i.e., a daily dose of 12 mg) may receive 12 mg/day or may instead receive 24 mg every 2 days, or 36 mg every 3 days, etc. In another illustrative embodiment, an individual (e.g. child) of 20 kilograms may receive the same daily dose of 6-TG (0.1 mg/kg/body weight) equating to 2 mg/day, or 4 mg/2 days or 6 mg/3 days, etc.

Accordingly, in certain embodiments the composition of the present invention comprising 6-TG comprises between about 0.1 mg/kg body weight and 2.5 mg/kg body weight of 6-TG. Suitably the composition comprises about 0.1 mg/kg body weight and 2.0 mg/kg body weight of 6-TG. Suitably the composition comprises about 0.1 mg/kg body weight and 1.5 mg/kg body weight of 6-TG. Suitably the composition comprises about 0.1 mg/kg body weight and 1.0 mg/kg body weight of 6-TG. Suitably the composition comprises about 0.1 mg/kg body weight and 0.8 mg/kg body weight of 6-TG. Suitably the composition comprises about 0.1 mg/kg body weight and 0.5 mg/kg body weight of 6-TG. Suitably the composition comprises about 0.3 mg/kg body weight and 2.5 mg/kg body weight of 6-TG. Suitably the composition comprises between 0.4 mg/kg body weight and 1 mg/kg body weight of 6-TG, more suitably the composition comprises between 0.5 mg/kg body weight and 1.0 mg/kg body weight of 6-TG, and even more suitably the composition comprises between 0.5 mg/kg body weight and 0.8 mg/kg body weight of 6-TG.

The precise dosage of 6-TG in the compositions of the present invention can depend on a variety of factors, such as the disease or condition to be treated, the progression or stage of the disease or condition, and/or the individual to be treated, including the age, gender, height, and/or weight of the individual. It is expected that the precise dosage can be determined by a practitioner in the art. In determining the dosage, the practitioner may evaluate the severity of the disease or condition, or the severity of the symptoms of the disease or condition, the individual's clinical history and responsiveness to previous therapies including any history of prior relapse of the disease or condition. It is expected that the dosage may be readily determined without undue experimentation.

### EXAMPLES

### Example 1: Preparation of 10 mg 6-TG Oral Formulation

### Core Formulation

10 mg polyethylene oxide having a molecular weight of about 2,000,000 g/mol (PEO 2M) was added to 10 mg of 6-TG and blended for 5 minutes. 336.39 mg PEO 2M was then added to the mixture and blended for 5 minutes. 3.6 mg magnesium stearate was then added to the mixture and blended for 2 minutes.

The final w/w % amounts of components in the core is set out in Table 1. The mixture was then compressed to form the core (Formulation 1).

### Coating

The core was coated with a mixture of Evonik Eudragit L30 D-55, PlasAcryl HTP20 and water as set out in Table 1.

Evonik Eudragit L30 D-55 is a premixed aqueous dispersion of methacrylic acid—ethyl acrylate copolymer having a molecular weight of about 320,000 g/mol.

PlasAcryl HTP20 is a premixed aqueous plasticiser comprising an anti-tacking agent (glycerol monostearate), a plasticiser (triethyl citrate) and a surfactant (polysorbate 80).

To coat the core formulation, 570 g of Evonik Eudragit L30 D-55 aqueous dispersion was dispensed into a vessel containing 284.5 g of purified water and stirred using an overhead mixer. To this dispersion was added 145.5 g PlasAcryl HTP20 under continuous stirring. The mixture was allowed to stir for 30 minutes prior to being sieved into another vessel ready for application. Tablets were placed in the coating pan and the pan rotated at 15-45 rpm. During this, the tablets were heated to between 30-40 degrees C. under a flow of hot air. The coating mixture was pumped from the stirred vessel to a spray nozzle situated about 10 cm above the tablets. Pressurized air was used to atomize the coating mixture producing a fine spray that coats the rotating tablets. The coating was applied until the tablet has gained approximately 10% w/w.

In another example, the compressed core was coated with a mixture of Colorcon Acryl-EZE 93F 19255, a plasticiser (triethyl citrate) and optionally an antifoam agent (simethicone). Acryl-EZE 93F 19255 is a premixed powder for aqueous dispersion. AcrylEZE is a ready-to-use mixture containing methacrylic acid copolymer, talc, optionally pigments, polyethylene glycol, silica, sodium bicarbonate, and sodium lauryl sulfate.

TABLE 1
Exemplary 6-TG oral formulation 1

| | |
|---|---|
| Ingredient | % w/w |

Core Components

| | |
|---|---|
| Thioguanine partial hemihydrate | 2.94* |
| Poly(ethylene oxide) WSR | 96.06 |
| N60K (PEO 2M) | |
| Magnesium stearate | 1.00 |
| Total | 100.00 |

Coating Components 1

| | |
|---|---|
| Evonik Eudragit L30 D-55^{&} | 57.00 |
| PlasAcryl HTP20^{&} | 14.55 |
| Water | 28.45 |

Coating Components 2 (alternative to 1)

| | |
|---|---|
| Acryl-EZE 93F19255 Clear | 15 |
| Triethyl citrate | 2 |
| Water | 83 |

*API quantity may be subject to correction for water of crystallisation, for example use of partial hemihydrate

### Example 2: In Vitro Dissolution Testing Procedure

### 2.1 Equipment Parameters

The UV-Vis Spectrophotometer System parameters are shown in Table 2.

TABLE 2
UV-Vis Spectrophotometer system

| | |
|---|---|
| Cuvettes | 5 mm |
| Wavelength of interest | 341 nm (±1 nm), i.e., |
| for fixed wavelength | the wavelength of |
| measurements | maximum absorbance |
| UV-Vis source | 370.0 nm |
| changeover | |

The dissolution bath system parameters are shown in Table 3.

TABLE 3
Dissolution bath system

| | |
|---|---|
| Apparatus | Baskets (Apparatus 1) |
| Medium volume at the | 900 mL (900 g) |
| end of dissolution: | |
| Temperature | 37 ± 0.5° C. |
| Speed | 100 ± 4 rpm |
| Sampling volume | 20 mL |

### 2.2 General Dissolution Test Procedure

The procedure followed was as follows:
1. Place one tablet into each basket.
2. Perform the dissolution test using the specified equipment parameters.
3. At the sampling time point, withdraw 20 mL of sample from each vessel through a cannula filter. When performing a dissolution profile, replace the same withdrawn volume with dissolution medium to maintain the volume in each vessel.
4. Filter each sample immediately through a syringe filter.

Using 5 mm quartz cuvettes, perform UV analysis as follows:
1. Acquire the spectra of the following solutions from 291 to 391 nm (i.e. 341±50 nm):
   Blank (Filtered dissolution medium) vs. blank
   Check standard working solution vs. blank
   Dissolution sample vs. blank
2. Under the photometric mode, analyse the following solutions at the wavelength of maximum absorbance of the check standard working solution (i.e. 341±1 nm as determined from the spectrum):
   Blank vs. blank
   Blank vs. air
   Check standard working solution vs. blank
   Calibration standard working solution vs. blank
   Dissolution sample solutions vs. blank
   Calibration standard working solution vs. blank
   Blank vs. blank

Calculate the % dissolution for each sample using the calculations set out below.

### Procedure

The system suitability acceptance criteria were:

### Spectral Analysis:

1. The blank vs. blank spectrum is within ±0.005 AU across the wavelength range of 291 to 391 nm.
2. The Amax of the check standard working solution should be 341±1 nm.
3. The Amax of the check standard working solution and sample solution should differ by not more than 2 nm.

### Fixed Wavelength Measurements:

1. The blank vs. blank readings are within 0.000 AU±0.005 AU at the beginning and end of analysis.
2. The blank vs. air reading is not more than 0.4 AU.
3. The % difference in the response factors of the check and calibration standard working solutions is not more than 2.0%.
4. The % difference between readings of the calibration standard working solution at the beginning and end of analysis is not more than 2.0%.

### Calculations for % Dissolution

To calculate the % dissolution for each sample, the following calculations were used:

$$Response factor = \frac{Standard Absorbance}{Standard mass}$$

Calculate the percentage of thioguanine dissolved in each sample using the following formula:

$$\begin{matrix}\% Dissolution = \frac{{AU}_{sa} \times std . m \times P \times 5 \times V \times 100}{{AU}_{std} \times 100 \times 50 \times LC} \\ = \frac{{AU}_{sa} \times std . m \times P \times V \times 0.1}{{AU}_{std} \times LC}\end{matrix}$$

where:
AUₛₐ=Absorbance of sample solution
AU_{std}=Absorbance of calibration standard working solution at the start of analysis
std.m=mass of thioguanine standard in calibration standard stock solution (mg)
P=% purity of thioguanine standard “as is”=100
LC=label claim (i.e. 10 mg/tablets)
V=volume of dissolution, for example 900 mL for dissolution carried out as described below at a single pH, or
for volume carried out at 3 different pHs (1.1, 6.5 and 6.8) as described below, the volume of dissolution medium at each stage (mL):
   Acid Stage (pH 1.1): 688 mL
   Buffer Stage 1 (pH 6.5): 880 mL
   Buffer Stage 2 (pH 6.8): 900 mL
for volume carried out at 3 different pHs as described below, the volume of dissolution medium at each stage (mL):
   Acid Stage (pH 1.1): 650 mL
   Buffer Stage 1 (pH5.5): 806 mL
   Buffer Stage 2 (pH 6.8): 900 mL

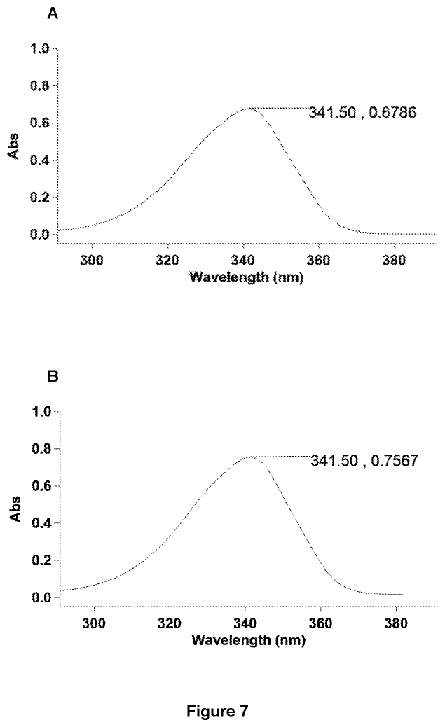

Examples of a typical Thioguanine Standard UV Spectrum and a Typical Sample Solution UV Spectrum are shown in FIG. 7.

The calculation to correct the percentage of thioguanine dissolved in each sample as shown in Table 4 below.

TABLE 4
Calculation for corrected percentage of thioguanine dissolved in
each sample

| Sampling Time | Corrected % Dissolution |
|---|---|
| 1 | a |
| 2 | $b + \frac{{x}_{3}}{{v}_{3 }} ( a )$ |
| 3 | $c + \frac{{x}_{1}}{{x}_{1}} ( a ) + \frac{{x}_{2}}{{v}_{2}} ( b )$ |
| v | v |
| [symbol] | [symbol] |
| [symbol] | [symbol] |
| 10 | $\begin{matrix}j + \frac{{x}_{1}}{{v}_{1}} ( a ) + \frac{{x}_{2}}{{v}_{2}} ( b ) + \frac{{x}_{3}}{{v}_{3}} ( c ) + \frac{{x}_{4}}{{v}_{4}} ( d ) + \frac{{x}_{5}}{{v}_{5}} ( e ) + \\ \frac{{x}_{6}}{{v}_{6}} ( f ) + \frac{{x}_{7}}{{v}_{7}} ( g ) + \frac{{x}_{8}}{{v}_{8}} ( h ) + \frac{{x}_{9}}{{v}_{9}} ( i )\end{matrix}$ |

[symbol]

indicates data missing or illegible when filed

where:
a, b, c, etc.=uncorrected dissolution results according to the formula in the method
xₙ=aliquot sampled (mL) at the time point n
vₙ=volume of dissolution medium (mL) per dissolution vessel at the time point n

### Preparation of Check and Calibration Solutions

To make Check Standard Stock Solution or Calibration Standard Stock Solution (both 100 μg/mL Thioguanine), weigh about 10 mg of Thioguanine reference standard and transfer into a 100 mL volumetric flask. Pipette 5.0 mL of DMSO into the flask, swirl to mix and allow to stand for 20 minutes so that active is visibly dissolved, swirl every 5 minutes. Add about 80 mL of dissolution medium and swirl to mix. Equilibrate to room temperature before diluting to volume with dissolution medium, mix well by inversion.

To make Check Standard Working Solution or Calibration Standard Working Solution (both 10 g/mL Thioguanine), pipette 5.0 mL of check standard stock solution into a 50 mL volumetric flask. Dilute to volume with dissolution medium, mix well by inversion.

### 2.3.1 Dissolution Test at Single pH

Dissolution testing was carried out at a single pH using a pH 6.8 phosphate buffer. The buffer solutions was prepared as follows:

### Prepare Dissolution Medium (pH 6.8 Phosphate Buffer)

To prepare 10 L: Mix 2500 mL of 0.2 M KH₂PO₄ solution with 1120 mL of 0.2 M NaOH solution. Dilute to 9 L with purified water and mix. Determine pH and if necessary, adjust using 0.2 M NaOH solution until pH 6.8±0.05. Make up to 10 L using purified water. Mix well. Measure and record the pH of the medium. Warm to about 40° C., filter and degas.

### 2.3.2 Dissolution Test at Increasing pH (Example at pH 1.1, 6.5 and 6.8)

Dissolution testing was also carried out increasing pH using three dissolution mediums.

For dissolution testing at pH 1.1, 6.5 and 6.8, the buffer and calibration solutions were prepared as follows:

### Dissolution Medium 1 (Acid Stage) (0.1 M HCl)

To prepare 10 L of 0.1 M HCl: dilute 85 mL of hydrochloric acid to 10 L with filtered and degassed purified water.

### Dissolution Medium 2 (Buffer Stage 1): Phosphate Buffer pH 6.50±0.05

To prepare 1 L: mix 688 ml of 0.1 M HCl with 192 mL of 0.2 M tri-sodium phosphate solution. If necessary, adjust pH to 6.50±0.05.

### Dissolution Medium 3 (Buffer Stage 2): Phosphate Buffer pH 6.80±0.05

To prepare 1 L: mix 688 ml of 0.1 M HCl with 212 mL of 0.2 M tri-sodium phosphate solution. If necessary, adjust pH to 6.80±0.05.

### Diluent

0.01 M NaOH Solution. Weigh 8.0 g of sodium hydroxide into a 1000 mL volumetric flask, add 600 mL purified water. Allow to cool to room temperature before making up to volume with purified water. Further dilute 50 mL of the resulting solution to 1000 mL with purified water.

### Blank Solutions

Blank: Dissolution Medium 3 (Phosphate buffer pH 6.80)
Blank 1: Dissolution Medium 1 (0.1 M HCl)
Blank 2: Dissolution Medium 2 (Phosphate buffer pH 6.50)

### Procedure Used for Increasing pH Dissolution Testing (at pH 1.1, 6.5 and 6.8)

For the dissolution testing at increasing pH, there were three stages including acid stage, buffer stage 1 and buffer stage 2. The sampling time points and replacement media for each stage are listed in Table 5 below.

TABLE 5
Sampling time points and replacement media for
each stage of increasing pH dissolution testing

| Stage | Time point (hours) | Media for replacement |
|---|---|---|
| Acid | 1, 2 | 0.1M HCl (Dissolution |
| stage | | Medium 1) |
| Buffer | 3 | Phosphate buffer pH 6.50 |
| stage 1 | | (Dissolution Medium 2) |
| Buffer | 4, 6, 8, 10, 12, 14, 17, | Phosphate buffer pH 6.80 |
| stage 2 | 20, 23, 26, 29, 32 and | (Dissolution Medium 3) |
| | 36 (or as required) | |

### Acid Stage

1. Add 688 mL of filtered and degassed 0.1 M HCl (Dissolution Medium 1) into each dissolution vessel and equilibrate to 37±0.5° C.
2. Place one tablet into each basket and start basket rotations.
3. After 2 hours operation in 0.1 M HCl, proceed immediately to buffer stage 1.

### Buffer Stage 1

1. With the basket operating at the specified rate in the work plan, add to the fluid in the vessel 192 mL of 0.2 M tri-sodium phosphate that has been equilibrate to 37±0.5° C. (complete the operation of adding the buffer within 5 mins).
2. Continue to operate for another 1 hour and then proceed to buffer stage 2.

### Buffer Stage 2

1. With the basket operating, add to the fluid in the vessel another 20 ml of 0.2 M tri-sodium phosphate that has been equilibrate to 37±0.5° C. (complete the operation of adding the buffer within 5 mins).
2. Continue to operate until the completion of dissolution.

At each specified sampling time point, withdraw 20 mL of sample from each vessel through the 35 μm polyethylene sipper filters into test tubes, replacing the sample amount taken with an equivalent amount of appropriate dissolution media.

### 2.3.3 Dissolution Test at Increasing pH (Example at pH 1.1, 5.5 and 6.8)

For dissolution testing at pH 1.1, 5.5 and 6.8, the buffer and calibration solutions were prepared as follows:

### Dissolution Medium 1 (Acid Stage) (0.1 M HCl)

0.1 M HCl (SS585-08) required volume approx. 4020 mL, i.e., 650 mL×6=3900 mL for stage 1 and an additional 120 mL for replacement. Solution should be prepared using filtered and degassed purified water.

### Dissolution Medium 2 (Buffer Stage 1 Replacement) (Phosphate Buffer pH 5.50)

To prepare 300 mL: Mix 242 mL of 0.1 HCl with 58 mL of 0.2 M tribasic-sodium phosphate solution. Adjust pH to 5.50±0.05 with either 0.1 M HCl or 0.2 M tribasic sodium phosphate. Degas by sonication.

### Dissolution Medium 3 (Buffer Stage 2 Replacement) (Phosphate Buffer pH 6.80)

To prepare 1700 mL: Mix 1300 mL of 0.1 M HCl with 400 mL of 0.2 M tribasic-sodium phosphate solution. Adjust pH to 6.80±0.05 with either 0.1 M HCl or 0.2 M tribasic-sodium phosphate solution. Degas by sonication.

### Solution A (For Buffer Stage 2)

To prepare 940 mL: Combine 440 mL of 0.2 M tribasic-sodium phosphate solution with 500 mL of purified water. Degas by sonication.

### Blank Solution

Dissolution medium 3 (Phosphate buffer pH 6.80).

Other pH dissolution mediums may be used as necessary to test different pHs.

### Procedure Used for Increasing pH Dissolution Testing (at pH 1.1, 5.5 and 6.8)

For the dissolution testing at increasing pH, there were three stages including acid stage, buffer stage 1, and buffer stage 2. The sampling time points and replacement media for each stage are listed in Table 6 below.

TABLE 6
Sampling time points and replacement media for
each stage of increasing pH dissolution testing

| Stage | Time Point (hours) | Media for Replacement |
|---|---|---|
| Acid | 1, 2 | Dissolution medium 1 |
| stage | | (0.1M HCl) |
| Buffer | 3 | Dissolution medium 2 |
| stage 1 | | (Phosphate buffer pH 5.5) |
| Buffer | 4, 6, 8, 10, 12, 14, 17, | Dissolution medium 3 |
| stage 2 | 20, 23, 26, 28 and 30 | (Phosphate buffer pH 6.8) |
| | (or as required) | |

### Acid Stage

1. Add 650 mL of filtered and degassed dissolution medium 1 into each dissolution vessel and equilibrate to 37±0.5° C. in the dissolution bath.
2. Place one tablet into each basket and start the basket rotations.
3. At the specified sampling time point (i.e., 1 or 2 hours), withdraw 20 mL of sample from each vessel through the permitted cannula filter. Replace the same withdrawn volume with dissolution medium 1 immediately following sampling, to maintain the volume in each vessel.
4. Immediately after sampling, filter aliquots of each sample into an appropriate test tube, passing through the permitted syringe filter and applying the prescribed discard volume.
5. Proceed immediately to buffer stage 1.

### Buffer Stage 1 (pH 5.5)

1. Without halting the instrument, add to the volume in each vessel 156 mL of 0.2 M tribasic-sodium phosphate solution which has been degassed and equilibrated to 37±0.5° C.
Note: Addition of the buffer should be completed within 5 minutes.
2. At the specified sampling time point (i.e., 3 hours), withdraw 20 mL of sample from each vessel through the permitted cannula filter. Replace the same withdrawn volume with dissolution medium 2 immediately following sampling, to maintain the volume in each vessel.
3. Immediately after sampling, filter aliquots of each sample into an appropriate test tube, passing through the permitted syringe filter and applying the prescribed discard volume.
4. Proceed immediately to buffer stage 2.

### Buffer Stage 2 (pH 6.8)

1. Without halting the instrument, add to the volume in each vessel 94 mL of solution A which has been degassed and equilibrated to 37±0.5° C.
Note: Addition of solution A should be completed within 5 minutes.
2. At the specified sampling time point (i.e., 4, 6, 8, 10, 12, 14, 17, 20, 23, 26, 28 and 30 hours), withdraw 20 mL of sample from each vessel through the permitted cannula filter. Replace the same withdrawn volume with dissolution medium 3 immediately following sampling, to maintain the volume in each vessel.
3. Immediately after sampling, filter aliquots of each sample into an appropriate test tube, passing through the permitted syringe filter and applying the prescribed discard volume.

### Example 3: Preparation and Testing of 10 mg 6-TG Core Formulations

Various core formulations were made as shown in Table 7. When formed into tablets with weights as shown in the table, the dosage is 10 mg 6-TG per tablet.

TABLE 7
Exemplary 6-TG core formulations

| | Amount % w/w | | | |
|---|---|---|---|---|
| | Core formulation no. | | | |
| Ingredient | 1 | 2 | 3 | 4 |
| Thioguanine | 2.78* | 2.78 | 2.78 | 3.08 |
| POLYOX ™ | 96.22 | | | 37.96 |
| WSR N-60K | | | | |
| (polyethylene | | | | |
| oxide 2,000,000) | | | | |
| POLYOX ™ | | 96.22 | | 37.96 |
| WSR-303 | | | | |
| (polyethylene | | | | |
| oxide, 7,000,000) | | | | |
| POLYOX ™ | | | 96.22 | |
| WSR 1105 | | | | |
| (polyethylene | | | | |
| oxide (900,000) | | | | |
| Magnesium | 1.00 | 1.00 | 1.00 | 1.00 |
| stearate | | | | |
| Ethocel std | | | | 20.00 |
| 100 prem | | | | |
| Total | 100.00 | 100.00 | 100.00 | 100.00 |
| | Tablet | Tablet | Tablet | Tablet |
| | weight | weight | weight | weight |
| | 360 mg | 360 mg | 360 mg | 325 mg |
| | for 10 mg | for 10 mg | for 10 mg | for 10 mg |
| | dose of | dose of | dose of | dose of |
| | 6-TG | 6-TG | 6-TG | 6-TG |

### Example 4: In Vitro Dissolution Testing of Formulation 1

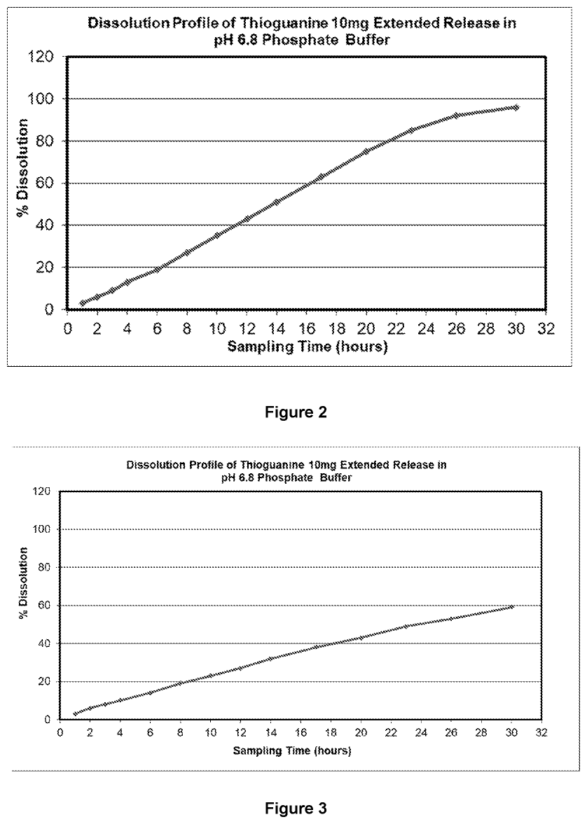

The release rate for core formulation 1 was tested at pH 6.8 over a period 30 hours (3 tablets, each with 10 mg of 6-TG). The cumulative percentage release of 6-TG over 30 hours for core formulation 1 is provided in Table 8 and summarized in graphical form in FIG. 2.

The release profile is approximately zero-order (i.e. linear) over about 1 to 23 hours, with a release rate of about 3-4 percent per hour. Close to 100% dissolution is reached at about 30-32 hours.

TABLE 8
In vitro dissolution profile of Core Formulation 1 a pH 6.8 over 30 hours

| | Sampling number and time (hr) | | | | | | | | | | | | | |
|---|---|---|---|---|---|---|---|---|---|---|---|---|---|---|
| | 1st | 2nd | 3rd | 4th | 5th | 6th | 7th | 8th | 9th | 10th | 11th | 12th | 13th | 14th |
| | 1 | 2 | 3 | 4 | 6 | 8 | 10 | 12 | 14 | 17 | 20 | 23 | 26 | 30 |
| | As | | | | | | | | | | | | | |
| Sample 1 | 0.0255 | 0.0469 | 0.0717 | 0.1001 | 0.1448 | 0.2010 | 0.2545 | 0.3111 | 0.3670 | 0.4492 | 0.5322 | 0.5982 | 0.6440 | 0.6566 |
| Sample 2 | 0.0229 | 0.0427 | 0.0659 | 0.0895 | 0.1441 | 0.1963 | 0.2579 | 0.3103 | 0.3668 | 0.4607 | 0.5429 | 0.6057 | 0.6383 | 0.6447 |
| Sample 3 | 0.0227 | 0.0446 | 0.0697 | 0.0904 | 0.1444 | 0.2016 | 0.2601 | 0.3156 | 0.3707 | 0.4662 | 0.5531 | 0.6170 | 0.6598 | 0.6793 |
| | % Dissolution (un-corrected) | | | | | | | | | | | | | |
| Sample 1 | 3.71 | 6.52 | 9.79 | 13.52 | 19.40 | 26.80 | 33.84 | 41.28 | 48.63 | 59.45 | 70.37 | 79.05 | 85.07 | 86.73 |
| Sample 2 | 3.37 | 5.98 | 9.03 | 12.14 | 19.32 | 26.19 | 34.30 | 41.20 | 48.64 | 60.99 | 71.81 | 80.08 | 84.37 | 85.21 |
| Sample 3 | 3.32 | 6.33 | 9.48 | 12.19 | 19.25 | 26.74 | 34.40 | 41.66 | 48.87 | 61.37 | 72.75 | 81.11 | 86.71 | 89.27 |
| | % Dissolution (Corrected) | | | | | | | | | | | | | |
| Sample 1 | 4 | 7 | 10 | 14 | 20 | 28 | 36 | 44 | 52 | 64 | 76 | 86 | 94 | 98 |
| Sample 2 | 3 | 6 | 9 | 13 | 20 | 27 | 36 | 44 | 52 | 65 | 78 | 87 | 94 | 96 |
| Sample 3 | 3 | 6 | 10 | 13 | 20 | 28 | 36 | 44 | 52 | 66 | 79 | 89 | 96 | 100 |
| Average | 3 | 6 | 10 | 13 | 20 | 28 | 36 | 44 | 52 | 65 | 78 | 87 | 95 | 98 |
| Release | | 3 | 4 | 3 | 3.5 | 4 | 4 | 4 | 4 | 4.3 | 4.3 | 3 | 2.6 | 0.75 |
| rate* | | | | | | | | | | | | | | |
| (%/hr) | | | | | | | | | | | | | | |
| Release | | 0.3 | 0.4 | 0.43 | 0.35 | 0.4 | 0.4 | 0.4 | 0.4 | 0.43 | 0.43 | 0.3 | 0.20 | 0.075 |
| rate* | | | | | | | | | | | | | | |
| (mg/hr) | | | | | | | | | | | | | | |

*Release rate from previous measurement

### Example 5: In Vitro Dissolution Testing of Formulation 2

The release rate for core formulation 2 was tested at pH 6.8 over a period 30 hours (3 tablets, each with 10 mg of 6-TG). The cumulative percentage release of 6-TG over 30 hours for core formulation 2 is provided in Table 9 and summarized in graphical form in FIG. 3.

The release profile is approximately zero-order (i.e. linear) over about 30 hours, with an R² valve of 0.987. However, the R² valve over hours 1-17 was 0.991, showing the release profile was more linear over the initial hours 1-17.

There was release rate between measurements was about 2% per hour. Only about 60% dissolution is reached at about 30 hours. Due to the lower percentage release (e.g. very extended release), there may be concern that in vivo the active could be excreted into municipal wastewater.

TABLE 9
In vitro dissolution profile of Core Formulation 2 at pH 6.8 over 30 hours

| | Sampling number and time (hr) | | | | | | | | | | | | | |
|---|---|---|---|---|---|---|---|---|---|---|---|---|---|---|
| | 1st | 2nd | 3rd | 4th | 5th | 6th | 7th | 8th | 9th | 10th | 11th | 12th | 13th | 14th |
| | 1 | 2 | 3 | 4 | 6 | 8 | 10 | 12 | 14 | 17 | 20 | 23 | 26 | 30 |
| | As | | | | | | | | | | | | | |
| Sample 1 | 0.0222 | 0.0388 | 0.0559 | 0.0697 | 0.1028 | 0.1344 | 0.1638 | 0.1913 | 0.2188 | 0.2588 | 0.2935 | 0.3239 | 0.3535 | 0.3923 |
| Sample 2 | 0.0221 | 0.0399 | 0.0564 | 0.0737 | 0.1086 | 0.1420 | 0.1744 | 0.2079 | 0.2393 | 0.2842 | 0.3223 | 0.3768 | 0.3796 | 0.4142 |
| Sample 3 | 0.0215 | 0.0380 | 0.0530 | 0.0686 | 0.0997 | 0.1292 | 0.1577 | 0.1839 | 0.2103 | 0.2506 | 0.2849 | 0.3219 | 0.3538 | 0.3968 |
| | % Dissolution (un-corrected) | | | | | | | | | | | | | |
| Sample 1 | 3.31 | 5.52 | 7.79 | 9.63 | 14.03 | 18.23 | 22.14 | 25.80 | 29.46 | 34.77 | 39.39 | 43.43 | 47.37 | 52.53 |
| Sample 2 | 3.31 | 5.69 | 7.89 | 10.20 | 14.86 | 19.32 | 23.65 | 28.12 | 32.31 | 38.31 | 43.40 | 50.67 | 51.05 | 55.67 |
| Sample 3 | 3.23 | 5.33 | 7.44 | 9.53 | 13.68 | 17.62 | 21.43 | 24.93 | 28.46 | 33.84 | 38.42 | 43.37 | 47.63 | 53.37 |
| | % Dissolution (Corrected) | | | | | | | | | | | | | |
| Sample 1 | 3 | 6 | 8 | 10 | 15 | 19 | 23 | 28 | 32 | 38 | 43 | 48 | 53 | 59 |
| Sample 2 | 3 | 6 | 8 | 11 | 15 | 20 | 25 | 30 | 35 | 42 | 47 | 56 | 57 | 63 |
| Sample 3 | 3 | 5 | 8 | 10 | 14 | 18 | 23 | 27 | 31 | 37 | 42 | 48 | 53 | 60 |
| Average | 3 | 6 | 8 | 10 | 15 | 19 | 24 | 28 | 33 | 39 | 44 | 51 | 54 | 61 |
| Release | | 3 | 2 | 2 | 2.5 | 2 | 2.5 | 2 | 2.5 | 2 | 1.7 | 2.3 | 1 | 1.5 |
| rate* | | | | | | | | | | | | | | |
| (%/hr) | | | | | | | | | | | | | | |
| Release | | 0.3 | 0.2 | 0.2 | 0.25 | 0.2 | 0.25 | 0.2 | 0.25 | 0.2 | 0.17 | 0.23 | 0.1 | 0.15 |
| rate* | | | | | | | | | | | | | | |
| (mg/hr) | | | | | | | | | | | | | | |

*Release rate from previous measurement

### Example 6: In Vitro Dissolution Testing of Formulation 3

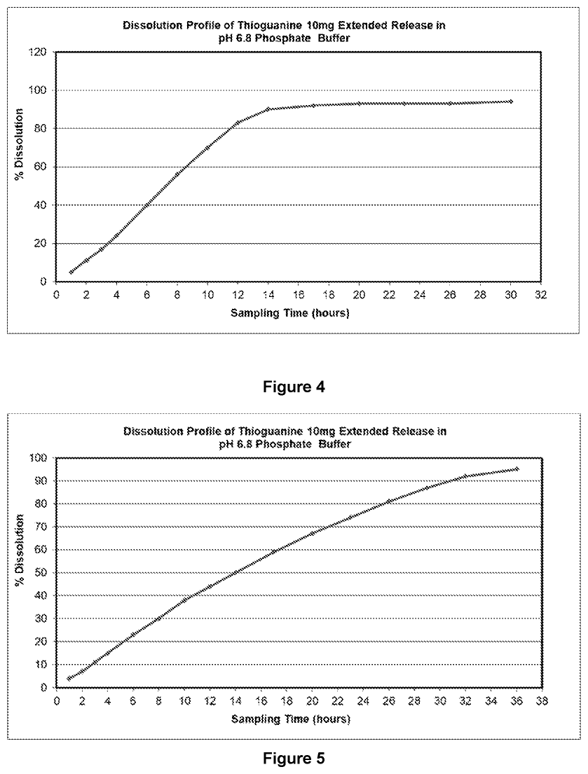

The release rate for core formulation 3 was tested at pH 6.8 over a period 30 hours (3 tablets, each with 10 mg of 6-TG). The cumulative percentage release of 6-TG over 30 hours for core formulation 3 is provided in Table 10 and summarized in graphical form in FIG. 4.

The release profile is approximately zero-order (i.e. linear) over about 1 to 12-14 hours, with an R² valve over 1-14 hours of 0.992 and an R² valve over 1-12 hours of 0.998. The R² valve over hours 1-30 was non-linear with an R² valve of 0.787, due to majority of release being in hours 1-14.

There was a release rate of about 6-8 percent per hour in hours 1-12. About 92% dissolution is reached at about 14 hours.

TABLE 10
In vitro dissolution profile of Core Formulation 3 at pH 6.8 over 30 hours

| | Sampling number and time (hr) | | | | | | | | | | | | | |
|---|---|---|---|---|---|---|---|---|---|---|---|---|---|---|
| | 1st | 2nd | 3rd | 4th | 5th | 6th | 7th | 8th | 9th | 10th | 11th | 12th | 13th | 14th |
| | 1 | 2 | 3 | 4 | 6 | 8 | 10 | 12 | 14 | 17 | 20 | 23 | 26 | 30 |
| | As | | | | | | | | | | | | | |
| Sample 1 | 0.0404 | 0.0820 | 0.1298 | 0.1808 | 0.2990 | 0.4235 | 0.5343 | 0.6278 | 0.6712 | 0.6763 | 0.6682 | 0.6546 | 0.6411 | 0.6279 |
| Sample 2 | 0.0391 | 0.0833 | 0.1342 | 0.1914 | 0.3109 | 0.4279 | 0.5312 | 0.6226 | 0.6495 | 0.6583 | 0.6526 | 0.6418 | 0.6294 | 0.6152 |
| Sample 3 | 0.0400 | 0.0838 | 0.1351 | 0.1925 | 0.3182 | 0.4365 | 0.5448 | 0.6361 | 0.6748 | 0.6823 | 0.6797 | 0.6640 | 0.6500 | 0.6360 |
| | % Dissolution (un-corrected) | | | | | | | | | | | | | |
| Sample 1 | 5.42 | 10.71 | 16.80 | 23.28 | 38.32 | 54.17 | 68.26 | 80.16 | 85.68 | 86.33 | 85.30 | 83.57 | 81.85 | 80.17 |
| Sample 2 | 5.28 | 10.94 | 17.45 | 24.77 | 40.06 | 55.03 | 68.24 | 79.94 | 83.38 | 84.50 | 83.77 | 82.39 | 80.81 | 78.99 |
| Sample 3 | 5.39 | 11.78 | 17.55 | 24.89 | 40.95 | 56.07 | 69.92 | 81.59 | 86.53 | 87.49 | 87.16 | 85.15 | 83.36 | 81.57 |
| | % Dissolution (Corrected) | | | | | | | | | | | | | |
| Sample 1 | 5 | 11 | 17 | 24 | 40 | 56 | 72 | 85 | 92 | 95 | 96 | 96 | 96 | 96 |
| Sample 2 | 5 | 11 | 18 | 26 | 41 | 57 | 72 | 85 | 90 | 93 | 94 | 95 | 95 | 95 |
| Sample 3 | 5 | 12 | 18 | 26 | 42 | 58 | 73 | 87 | 93 | 96 | 98 | 98 | 98 | 98 |
| Average | 5 | 11 | 18 | 25 | 41 | 57 | 72 | 86 | 92 | 95 | 96 | 96 | 96 | 96 |
| Release | | 6 | 7 | 7 | 8 | 8 | 7.5 | 7 | 3 | 1 | 0.3 | 0 | 0 | 0 |
| rate* | | | | | | | | | | | | | | |
| (%/hr) | | | | | | | | | | | | | | |
| Release | | 0.6 | 0.7 | 0.7 | 0.8 | 0.8 | 0.75 | 0.7 | 0.3 | 0.1 | 0.03 | 0 | 0 | 0 |
| rate* | | | | | | | | | | | | | | |
| (mg/hr) | | | | | | | | | | | | | | |

*Release rate from previous measurement

### Example 7: In Vitro Dissolution Testing of Formulation 4

The release rate for core formulation 4 was tested at pH 6.8 over a period 30 hours (3 tablets, each with 10 mg of 6-TG). The cumulative percentage release of 6-TG over 30 hours for core formulation 4 is provided in Table 11 and summarized in graphical form in FIG. 5.

The release profile was less linear than the above examples, with an R² valve over 1-36 hours of 0.952 and an R² valve over 1-29 hours of 0.971.

About 95% dissolution was reached at about 36 hours.

The release rate was about 3-4% per hour.

TABLE 11
In vitro dissolution profile of comparative Core Formulation 4 at pH 6.8 over 36 hours

| | Sampling number and time (hr) | | | | | | | | |
|---|---|---|---|---|---|---|---|---|---|
| | 1st | 2nd | 3rd | 4th | 5th | 6th | 7th | 8th | 9th |
| | 1 | 2 | 3 | 4 | 6 | 8 | 10 | 12 | 14 |
| | As | | | | | | | | |
| Sample 1 | 0.0328 | 0.0614 | 0.0880 | 0.1199 | 0.1745 | 0.2288 | 0.2831 | 0.3243 | 0.3732 |
| Sample 2 | 0.0316 | 0.0601 | 0.0864 | 0.1176 | 0.1688 | 0.2187 | 0.2741 | 0.3160 | 0.3613 |
| Sample 3 | 0.0285 | 0.0577 | 0.0855 | 0.1191 | 0.1727 | 0.2270 | 0.2816 | 0.3189 | 0.3589 |
| | % Dissolution (un-corrected) | | | | | | | | |
| Sample 1 | 3.77 | 7.28 | 10.54 | 14.45 | 21.15 | 27.82 | 34.48 | 39.53 | 45.54 |
| Sample 2 | 3.58 | 7.04 | 10.23 | 14.02 | 20.23 | 26.29 | 33.01 | 38.10 | 43.59 |
| Sample 3 | 3.77 | 5.88 | 11.90 | 16.70 | 24.35 | 32.10 | 39.89 | 45.21 | 50.92 |
| | % Dissolution (Corrected) | | | | | | | | |
| Sample 1 | 4 | 7 | 11 | 15 | 22 | 29 | 36 | 42 | 49 |
| Sample 2 | 4 | 7 | 10 | 14 | 21 | 28 | 35 | 41 | 47 |
| Sample 3 | 4 | 6 | 12 | 17 | 25 | 33 | 42 | 48 | 55 |
| Average | 4 | 7 | 11 | 15 | 23 | 30 | 38 | 44 | 50 |
| Release rate* | | 3 | 4 | 4 | 4 | 3.5 | 4 | 3 | 3 |
| (%/hr) | | | | | | | | | |
| Release rate* | | 0.3 | 0.4 | 0.4 | 0.4 | 0.35 | 0.4 | 0.3 | 0.3 |
| (mg/hr) | | | | | | | | | |

| | Sampling number and time (hr) | | | | | | |
|---|---|---|---|---|---|---|---|
| | 10th | 11th | 12th | 13th | 14th | 15th | 16th |
| | 17 | 20 | 23 | 26 | 29 | 32 | 36 |
| | As | | | | | | |
| Sample 1 | 0.4383 | 0.5020 | 0.5572 | 0.6000 | 0.6282 | 0.6486 | 0.6599 |
| Sample 2 | 0.4236 | 0.4761 | 0.5258 | 0.5675 | 0.6001 | 0.6307 | 0.6557 |
| Sample 3 | 0.4152 | 0.4562 | 0.4945 | 0.5331 | 0.5620 | 0.5819 | 0.5893 |
| | % Dissolution (un-corrected) | | | | | | |
| Sample 1 | 53.52 | 61.34 | 68.11 | 73.36 | 76.82 | 79.33 | 80.71 |
| Sample 2 | 51.15 | 57.53 | 63.56 | 68.62 | 72.58 | 76.29 | 79.32 |
| Sample 3 | 58.96 | 64.81 | 70.27 | 75.78 | 79.91 | 82.75 | 83.80 |
| | % Dissolution (Corrected) | | | | | | |
| Sample 1 | 58 | 67 | 75 | 82 | 87 | 91 | 94 |
| Sample 2 | 56 | 63 | 70 | 77 | 82 | 88 | 92 |
| Sample 3 | 64 | 71 | 78 | 85 | 91 | 96 | 99 |
| Average | 59 | 67 | 74 | 81 | 87 | 92 | 95 |
| Release rate* | 3 | 2.7 | 2.3 | 2.3 | 2 | 1.7 | 1 |
| (%/hr) | | | | | | | |
| Release rate* | 0.3 | 0.27 | 0.23 | 0.23 | 0.2 | 0.17 | 0.1 |
| (mg/hr) | | | | | | | |

*Release rate from previous measurement

### Example 8: Preparation and Testing of 10 mg 6-TG Oral Formulation

A further exemplary example of a 6-TG oral formulation was prepared. The formulation is given in Table 12.

The following method was used to prepare the tablets:
1. Thioguanine (21.98 g) and Poly(ethylene oxide) WSR N60K (40.52 g) were mixed in a container for 5 minutes.
2. Further Poly(ethylene oxide) WSR N60K (680 g) was weighed, sieved and transferred into the container and mixed for 10 minutes.
3. Magnesium stearate (7.50 g) was weighed, sieved and transferred into the container and mixed for 2 minutes.
4. Tablets of approximate weight 360 mg were compressed using a RIVA press using RISE tooling.
5. Potency can be adjusted for the use of hemihydrate Thioguanine or anhydrate. Potency 10 mg per tablet.

To coat the tablets the following process was performed:
1. A vessel was tared and the purified water and triethyl citrate dispensed into it.
2. The Acryl-EZE powder was added into the vessel and overhead mixing was applied for at least 30 minutes until suspension appeared uniform.
3. The suspension was passed through a mesh sieve prior to coating.
4. The tablets were loaded into a fluid bed drier, preheated to 60° C.
5. A test spray was performed with no tablets to ensure the suspension nozzle was compatible and the spray was adjusted as required.
6. Coating was began at a slow rate and increased throughout run. Tablet bed temperature was not allowed to exceed 65° C.
7. The total mass of the tablets was weighed after drying for 30 minutes following coating. The tablets were coated to a target weight gain of 10%, e.g. total weight of tablet×1.10.

TABLE 12
Exemplary 6-TG oral formulation

| | | Amount | Quantity |
|---|---|---|---|
| | | (% w/w) | (g) |
| | | Formulation no. | |
| Material | Purpose | 5 | 5 |
| Core Components | | | |
| Thioguanine | Active | 2.93 | 21.98 |
| | Pharmaceutical | | |
| | Ingredient | | |
| POLYOX WSR | Extended-release | 96.07 | 720.52 |
| N-60K (Polyethylene | matrix | | |
| oxide 2,000,000) | | | |
| Magnesium stearate | Tablet Lubricant | 1.00 | 7.50 |
| Total: | | 100 | 750 |
| Coating Components | | | |
| Acryl-EZE 93F | Enteric coating | 15 | 60.0 |
| 19255 | | | |
| Triethyl citrate | Plasticiser | 2 | 8.0 |
| Purified water | Enteric coating | 83 | 332.0 |

| | | |
|---|---|---|
| Total: | 100 | 400.0 |

### Example 9: In Vitro Dissolution Testing of 6-TG Oral Formulation-Formulation 5 with Enteric Coating

The formulation 5 was dissolution tested with hour 1-2 in pH 1.1, hour 3 in pH 6.5, and hours 4-36 in pH 6.8 according to the in vitro dissolution testing procedure in Example 2.

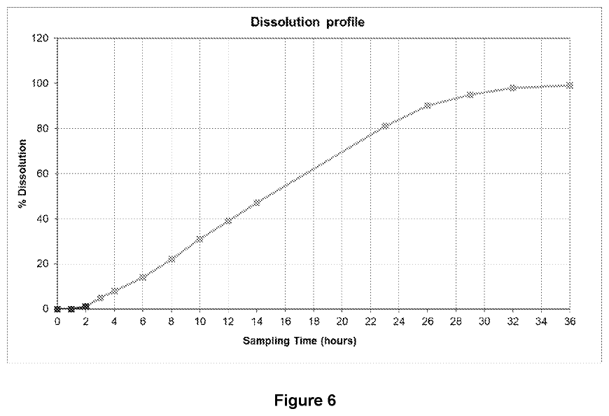

The cumulative percentage release of 6-TG over 36 hours for formulation is provided in Table 13 and summarized in graphical form in FIG. 6.

The release profile is approximately zero-order (i.e. approximately linear) over about 4 to 26 hours (at pH 6.8), with an R² of 0.994.

There was only about 1% dissolution after 2 hours at pH 1.1.

There was a release rate of about 3-4.5% per hour in hours 3-26.

About 98% dissolution is reached at about 32 hours.

TABLE 13
In vitro dissolution profile of Formulation 5 over 36 hours (hours 1-2 in pH 1.1, hour 3 in pH 6.5, hours 4-36 in pH 6.8)

| | Sampling time (hours) | | | | | | | | | | | | | |
|---|---|---|---|---|---|---|---|---|---|---|---|---|---|---|
| | 1 | 2 | 3 | 4 | 6 | 8 | 10 | 12 | 14 | 23 | 26 | 29 | 32 | 36 |
| | Au | | | | | | | | | | | | | |
| Sample | 0.0021 | 0.0051 | 0.0273 | 0.0526 | 0.1093 | 0.1667 | 0.2342 | 0.3004 | 0.3688 | 0.6026 | 0.6650 | 0.7069 | 0.7238 | 0.7143 |
| 1 | | | | | | | | | | | | | | |
| Sample | 0.0049 | 0.0131 | 0.0381 | 0.0637 | 0.1078 | 0.1677 | 0.2246 | 0.2876 | 0.3504 | 0.6292 | 0.6831 | 0.7054 | 0.6961 | 0.6865 |
| 2 | | | | | | | | | | | | | | |
| Sample | 0.0049 | 0.0140 | 0.0397 | 0.0639 | 0.1126 | 0.1713 | 0.2324 | 0.3008 | 0.3599 | 0.6489 | 0.6970 | 0.6990 | 0.6881 | 0.6754 |
| 3 | | | | | | | | | | | | | | |
| Sample | 0.0047 | 0.0130 | 0.0403 | 0.0664 | 0.1142 | 0.1737 | 0.2372 | 0.3013 | 0.3587 | 0.6014 | 0.6614 | 0.7074 | 0.7233 | 0.7122 |
| 4 | | | | | | | | | | | | | | |
| Sample | 0.0046 | 0.0127 | 0.0375 | 0.0610 | 0.1118 | 0.1613 | 0.2244 | 0.2788 | 0.3304 | 0.5716 | 0.6248 | 0.6599 | 0.6667 | 0.6590 |
| 5 | | | | | | | | | | | | | | |
| Sample | 0.0045 | 0.0117 | 0.0362 | 0.0606 | 0.1126 | 0.1687 | 0.2367 | 0.3009 | 0.3590 | 0.6134 | 0.6605 | 0.6897 | 0.6994 | 0.6917 |
| 6 | | | | | | | | | | | | | | |
| | % Dissolution (Uncorrected) | | | | | | | | | | | | | |
| Sample | 0.06 | 0.35 | 3.21 | 6.49 | 13.68 | 20.97 | 29.54 | 37.94 | 46.63 | 76.31 | 84.23 | 89.55 | 91.69 | 90.49 |
| 1 | | | | | | | | | | | | | | |
| Sample | 0.33 | 1.13 | 4.55 | 7.90 | 13.50 | 21.10 | 28.33 | 36.33 | 44.30 | 79.71 | 86.55 | 89.38 | 88.20 | 86.98 |
| 2 | | | | | | | | | | | | | | |
| Sample | 0.33 | 1.22 | 4.76 | 7.94 | 14.14 | 21.61 | 29.38 | 38.08 | 45.60 | 82.38 | 88.50 | 88.75 | 87.36 | 85.75 |
| 3 | | | | | | | | | | | | | | |
| Sample | 0.31 | 1.10 | 4.75 | 8.11 | 14.08 | 21.52 | 29.45 | 37.46 | 44.64 | 74.97 | 82.46 | 88.21 | 90.20 | 88.81 |
| 4 | | | | | | | | | | | | | | |
| Sample | 0.30 | 1.10 | 4.50 | 7.60 | 14.09 | 20.41 | 28.47 | 35.42 | 42.01 | 72.82 | 79.62 | 84.10 | 84.97 | 83.98 |
| 5 | | | | | | | | | | | | | | |
| Sample | 0.29 | 0.99 | 4.31 | 7.51 | 14.11 | 21.24 | 29.88 | 38.04 | 45.42 | 77.74 | 83.72 | 87.43 | 88.66 | 87.69 |
| 6 | | | | | | | | | | | | | | |
| | % Dissolution (Corrected) | | | | | | | | | | | | | |
| Sample | 0 | 0 | 3 | 7 | 14 | 22 | 31 | 40 | 49 | 80 | 89 | 97 | 101 | 102 |
| 1 | | | | | | | | | | | | | | |
| Sample | 0 | 1 | 5 | 8 | 14 | 22 | 29 | 38 | 47 | 83 | 92 | 97 | 97 | 98 |
| 2 | | | | | | | | | | | | | | |
| Sample | 0 | 1 | 5 | 8 | 14 | 22 | 31 | 40 | 48 | 86 | 94 | 96 | 97 | 97 |
| 3 | | | | | | | | | | | | | | |
| Sample | 0 | 1 | 5 | 8 | 14 | 22 | 31 | 39 | 47 | 79 | 88 | 95 | 99 | 100 |
| 4 | | | | | | | | | | | | | | |
| Sample | 0 | 1 | 5 | 8 | 14 | 21 | 30 | 37 | 45 | 76 | 85 | 91 | 94 | 95 |
| 5 | | | | | | | | | | | | | | |
| Sample | 0 | 1 | 4 | 8 | 14 | 22 | 31 | 40 | 48 | 81 | 89 | 95 | 98 | 99 |
| 6 | | | | | | | | | | | | | | |
| Average | 0 | 1 | 5 | 8 | 14 | 22 | 31 | 39 | 47 | 81 | 90 | 95 | 98 | 99 |
| Release | | 1 | 4 | 3 | 3 | 4 | 4.5 | 4 | 4 | 3.8 | 3 | 1.7 | 1 | 0.3 |
| rate* | | | | | | | | | | | | | | |
| (%/hr) | | | | | | | | | | | | | | |
| Release | | 0.1 | 0.4 | 0.3 | 0.3 | 0.4 | 0.45 | 0.4 | 0.4 | 0.38 | 0.3 | 0.17 | 0.1 | 0.03 |
| rate* | | | | | | | | | | | | | | |
| (mg/hr) | | | | | | | | | | | | | | |

*Release rate from previous measurement

### Example 10: Preparation of 10 mg 6-TG Oral Formulation

A further exemplary example of a 10 mg 6-TG oral formulation was prepared according to the same general method as Example 8. The formulation is given in Table 14.

TABLE 14
Exemplary 6-TG oral formulation

| | | | |
|---|---|---|---|
| | | Amount % | |
| Material | Purpose | w/w | mg/tablet |

Core Components

| | | | |
|---|---|---|---|
| Thioguanine | Active Pharmaceutical | 2.672 | 10.58 |
| hemihydrate | Ingredient | | |
| Polyethylene | Extended-release | 87.328 | 345.82 |
| oxide N60K | matrix | | |
| (PEO 2,000,000) | | | |
| Magnesium stearate | Tablet Lubricant | 0.909 | 3.600 |

Coating Components

| | | | |
|---|---|---|---|
| Triethyl citrate | Plasticiser | 1.071 | 31.760 |
| AcrylEZE powder | Enteric coating | 8.020 | 4.240 |
| (93F19255) | | | |
| | Total: | 100 | 396.000 |

### Example 11: In Vitro Dissolution Testing of 6-TG Oral Formulation of Example 10

The formulation of Example 10 was dissolution tested with hour 1-2 in pH 1.1, hour 3 in pH 5.5, and hours 4-30 in pH 6.8 according to the in vitro dissolution testing procedure in Example 2.

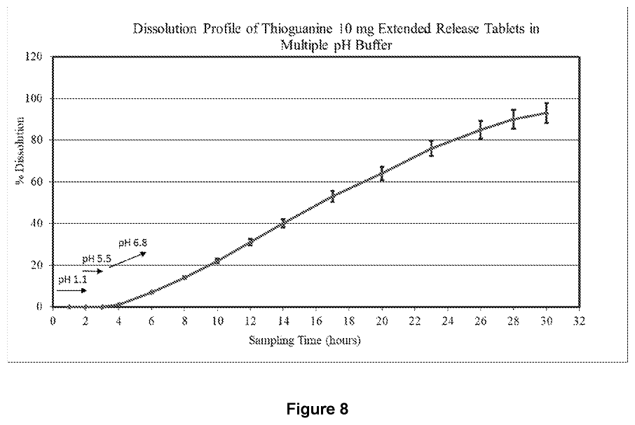

The cumulative percentage release of 6-TG over 30 hours for formulation is provided in Table 15 and summarized in graphical form in FIG. 8.

The release profile is approximately zero-order (i.e. approximately linear) over about 4 to 23 hours (at pH 6.8), with an R² of 0.995 and an R² of 0.994 over about 4 to 26 hours (at pH 6.8).

There was 0% dissolution after 2 hours at pH 1.1, and also 0% dissolution after 1 hour at pH 5.5.

There was a release rate of about 3-4.5% per hour in hours 3-26.

About 93% dissolution is reached at about 30 hours (26 hours at pH 6.8).

TABLE 15
In vitro dissolution profile of Formulation of Example 10 over 30
hours (hours 1-2 in pH 1.1, hour 3 in pH 5.5, hours 4-30 in pH 6.8)

| | Sampling time (hours) | | | | | | | | |
|---|---|---|---|---|---|---|---|---|---|
| | 1 | 2 | 3 | 4 | 6 | 8 | 10 | 12 | 14 |
| | Peak Area | | | | | | | | |
| Sample 1 | 0 | 0 | 0 | 2580 | 13271 | 27288 | 41775 | 57194 | 72747 |
| Sample 2 | 0 | 0 | 0 | 1588 | 11998 | 24017 | 37180 | 52023 | 67314 |
| Sample 3 | 0 | 0 | 0 | 1716 | 12875 | 25904 | 40982 | 57294 | 74311 |
| Sample 4 | 0 | 0 | 0 | 1951 | 12735 | 26490 | 41627 | 57172 | 72623 |
| Sample 5 | 0 | 0 | 0 | 1549 | 11792 | 24734 | 38651 | 53871 | 67776 |
| Sample 6 | 0 | 0 | 0 | 1372 | 12215 | 25028 | 39600 | 54256 | 68207 |
| | % Dissolution (Uncorrected) | | | | | | | | |
| Sample 1 | 0.00 | 0.00 | 0.00 | 1.39 | 7.15 | 14.69 | 22.49 | 30.80 | 39.17 |
| Sample 2 | 0.00 | 0.00 | 0.00 | 0.86 | 6.46 | 12.93 | 20.02 | 28.01 | 36.25 |
| Sample 3 | 0.00 | 0.00 | 0.00 | 0.92 | 6.93 | 13.95 | 22.07 | 30.85 | 40.01 |
| Sample 4 | 0.00 | 0.00 | 0.00 | 1.05 | 6.86 | 14.26 | 22.41 | 30.78 | 39.10 |
| Sample 5 | 0.00 | 0.00 | 0.00 | 0.83 | 6.35 | 13.32 | 20.81 | 29.01 | 36.49 |
| Sample 6 | 0.00 | 0.00 | 0.00 | 0.74 | 6.58 | 13.48 | 21.32 | 29.21 | 36.73 |
| | % Dissolution (Corrected) | | | | | | | | |
| Sample 1 | 0 | 0 | 0 | 1 | 7 | 15 | 23 | 32 | 41 |
| Sample 2 | 0 | 0 | 0 | 1 | 6 | 13 | 20 | 29 | 38 |
| Sample 3 | 0 | 0 | 0 | 1 | 7 | 14 | 23 | 32 | 42 |
| Sample 4 | 0 | 0 | 0 | 1 | 7 | 14 | 23 | 32 | 41 |
| Sample 5 | 0 | 0 | 0 | 1 | 6 | 13 | 21 | 30 | 38 |
| Sample 6 | 0 | 0 | 0 | 1 | 7 | 14 | 22 | 30 | 38 |
| Average | 0 | 0 | 0 | 1 | 7 | 14 | 22 | 31 | 40 |
| Release rate* | — | 0 | 0 | 1 | 3 | 3.5 | 4 | 4.5 | 4.5 |
| (%/hr) | | | | | | | | | |
| Release rate* | — | 0 | 0 | 0.1 | 0.3 | 0.35 | 0.4 | 0.45 | 0.45 |
| (mg/hr) | | | | | | | | | |

| | Sampling time (hours) | | | | | |
|---|---|---|---|---|---|---|
| | 17 | 20 | 23 | 26 | 28 | 30 |
| | Peak Area | | | | | |
| Sample 1 | 94771 | 112946 | 132299 | 146399 | 152010 | 154705 |
| Sample 2 | 89093 | 106718 | 125025 | 136658 | 142881 | 145039 |
| Sample 3 | 98820 | 119038 | 137850 | 151145 | 154818 | 156335 |
| Sample 4 | 95439 | 114580 | 132752 | 149139 | 154162 | 156250 |
| Sample 5 | 91648 | 112192 | 134007 | 148049 | 153982 | 153251 |
| Sample 6 | 91530 | 110756 | 130609 | 145075 | 151918 | 153137 |
| | % Dissolution (Uncorrected) | | | | | |
| Sample 1 | 51.03 | 60.82 | 71.24 | 78.83 | 81.85 | 83.30 |
| Sample 2 | 47.97 | 57.46 | 67.32 | 73.58 | 76.94 | 78.10 |
| Sample 3 | 53.21 | 64.10 | 74.23 | 81.39 | 83.36 | 84.18 |
| Sample 4 | 51.39 | 61.70 | 71.48 | 80.31 | 83.01 | 84.13 |
| Sample 5 | 49.35 | 60.41 | 72.16 | 79.72 | 82.91 | 82.52 |
| Sample 6 | 49.29 | 59.64 | 70.33 | 78.12 | 81.80 | 82.46 |
| | % Dissolution (Corrected) | | | | | |
| Sample 1 | 54 | 65 | 76 | 85 | 90 | 94 |
| Sample 2 | 50 | 61 | 72 | 80 | 85 | 88 |
| Sample 3 | 56 | 68 | 79 | 88 | 92 | 95 |
| Sample 4 | 54 | 65 | 77 | 87 | 91 | 94 |
| Sample 5 | 52 | 64 | 77 | 86 | 91 | 93 |
| Sample 6 | 52 | 63 | 75 | 85 | 90 | 92 |
| Average | 53 | 64 | 76 | 85 | 90 | 93 |
| Release rate* | 4.3 | 3.6 | 4 | 3 | 2.5 | 1.5 |
| (%/hr) | | | | | | |
| Release rate* | 0.43 | 0.36 | 0.4 | 0.3 | 0.25 | 0.15 |
| (mg/hr) | | | | | | |

*Release rate from previous measurement

### Example 12: Administration of 10 mg 6-TG Oral Formulation to Patient

A male subject (ethnicity white) in age bracket 60-70 in weight bracket 90-110 kg with a 15-year history of ulcerative colitis (UC), took one enterically coated 6-TG 10 mg extended-release oral tablet daily (formulation of Example 10) and mesalamine (2 g) over 3 months, followed by only the 6-TG 10 mg extended-release tablet over 7 months.

The subject was previously treated with mercaptopurine (50 mg to 75 mg alternate dose daily) and mesalamine (2 g daily). In the past, the subject took adalimumab (anti-TNF antibody) and previously required courses of steroid treatment to achieve remission.

One to two months prior to taking the 6-TG extended-release oral tablet (while being treated with the mercaptopurine/mesalamine) the subject had the following endoscopic and microscopy assessments:
Endoscopic assessment: Active disease consistent with Ulcerative Colitis was found from the rectum to the sigmoid colon. The Ulcerative Colitis Endoscopic Index of Severity (UCEIS) was V1E1B0 equal to 2/8, and the Mayo score was 1. The remainder of the ascending colon, colonic anastomosis and transverse colon all looked normal.
Microscopy: Assessment reveals colonic mucosa with shortening and branching of glands in association with diffuse inflammation comprising chronic inflammatory cells with moderate numbers of neutrophil. Regenerative epithelial changes were present. No pathogens, granulomas, dysplasia or malignancy was seen. In summary biopsies from the distal sigmoid colon showed Chronic Active Colitis.

Approximately 10 months after starting daily administration of extended release 6-TG tablets (and 7 months after starting monotherapy with the 6-TG) the subject had the following endoscopic and microscopy assessments:
Endoscopy summary: UCEIS V0 E0 B0=0/8, Mayo score 0 throughout the colon and rectum. There was one small patch with small apthous ulcers in the distal sigmoid colon only (Mayo 1, UCEIS V1 B0 E1=2/8).
Microscopy summary: Sections of rectal mucosa showed mild crypt distortion and Paneth cell metaplasia. There is no cryptitis, ulceration, granulomas, viral inclusions, dysplasia or evidence of malignancy. In summary biopsies showed Mild Chronic Proctitis.

Following the treatment with the extended release 6-TG oral tablet there was an improvement over the previous endoscopic and microscopy summaries. An improvement was shown even though the subject was previously being treated with mercaptopurine and mesalamine and even after seven months of monotherapy with the 6-TG extended-release tablet.

### Comparison of Thiopurine Metabolites in Red Blood Cells (RBC)

While taking 6-TG extended release tablets, the subject's metabolite levels were monitored with blood testing. 6-TGN and 6-MMP levels were consistently low. Examples of blood test results taken at two different time points are shown in Table 16.

TABLE 16
Blood test results monitoring thiopurine metabolite levels in subject

| Date of test | 6-TGN | 6-MMP |
|---|---|---|
| (months after | concentration | concentration |
| start of taking | in red blood | in red blood |
| extended-release | cells (RBC) | cells (RBC) |
| 6-TG) | (pmol/8 × 10(8)) | (pmol/8 × 10(8)) |
| About 5 months | 106 | <50 |
| About 7 months | 50 | |

For comparison, patients being treated for inflammatory bowel diseases with azathioprine or 6-mercaptopurine have blood test monitoring with dose adjustment aiming for 6-TGN concentrations from 235 to 450 pmol/8×10 (8) RBC. This range is to optimise efficacy whilst minimising risk for toxicity. Concentrations below 235 pmol/8×10 (8) RBC are associated with higher risk of treatment failure, whilst concentrations above 450 pmol/8×10 (8) RBC are associated with increased risk of leucopenia although this can sometimes occur at lower concentrations.

Patients on immediate release 6-thioguanine therapy for IBD (e.g. Thiosix™ tablets or Lanvis™ half tablets), who have an average dose of 20 mg daily, often have a 6-TGN level in the therapeutic interval of between 800 to 1200 pmol/8×10 (8) RBC.

6-MMP is generally accepted to be a toxic metabolite. RBC levels do not correlate with therapeutic efficacy. 6 MMP>5700 pmol/8×10 (8) RBC correlates with increased risk for hepatotoxicity. Low 6-TGN and markedly high 6-MMP (i.e. 6 MMP to 6TGN ratio>20) is associated with an increased risk of poor response to standard therapies.

Examples of the subject's 6-TGN and 6-MMP concentrations in RBC at various time points while they were being treated with mercaptopurine (previous treatment) are shown in Table 17.

TABLE 17
Blood test results while the subject was being
treated with mercaptopurine (for comparison)

| | Approx. timing | 6-TGN | 6-MMP |
|---|---|---|---|
| | of test (prior to | concentration | concentration |
| | taking extended | in red blood | in red blood |
| Drug being taken | release 6-TG | cells (RBC) | cells (RBC) |
| to treat UC | tablet) | (pmol/8 × 10(8)) | (pmol/8 × 10(8)) |
| Systemic | 11 years prior | 250 | 740 |
| mercaptopurine | | | |
| Systemic | 5 years prior | 675 | |
| mercaptopurine | | | |
| Systemic | 6 months prior | 585 | 401 |
| mercaptopurine | | | |

As shown in Table 16, the systemic levels of both 6-TGN and 6-MMP when treated with extended release 6-TG are low when compared to the normal therapeutic interval levels for standard 6-TG treatment (e.g. Thiosix™ tablets or Lanvis™ half tablets) and/or compared to normal therapeutic interval levels expected for azathioprine or 6-mercaptopurine treatment and/or compared to the subjects previous levels while being treated with mercaptopurine. This may be beneficial from a toxicity/safety perspective.

For comparison, in a 2022 paper in Inflammatory Bowel Diseases (“Rectally Administrated Thioguanine for Distal Ulcerative Colitis: A Multicenter Case Series, Crouwel et al. Inflammatory Bowel Diseases, 2022, XX, 1-5, https://doi.org/10.1093/ibd/izac195, Advance access publication 13 Sep. 2022) it was noted the median 6-TGN levels of 3 patients solely treated with thioguanine (20 mg) enemas were respectively 140, 153, and 315 pmol×10\hat{ }8 RBCs after a median of 33 days. The median 6-TGN levels of 7 patients solely treated with thioguanine (20 mg) suppositories was 32 pmol×10\hat{ }8 RBCs (IQR, 15-93) after a median of 15 days (IQR, 10-20). The 6-TGN concentration in RBC for the 6-TG 10 mg extended-release oral tablet was therefore closer to these locally administered treatments (i.e. suppository and enema) than previously known orally administered immediate release tablets.

1. A pharmaceutical composition comprising 6-thioguanine (6-TG), and a polyethylene oxide polymer having a molecular weight of between about 900,000 g/mol and about 9,000,000 g/mol.

2. The pharmaceutical composition according to claim 1, wherein the composition is formulated for oral administration.

3. The pharmaceutical composition according to claim 1, wherein the polyethylene oxide polymer has a molecular weight greater than about 900,000 g/mol and less than about 7,000,000 g/mol.

4. The pharmaceutical composition according to claim 1, wherein the polyethylene oxide polymer has a molecular weight of about 2,000,000 g/mol.

5. The pharmaceutical composition according to claim 1, wherein the polyethylene oxide polymer comprises substantially a single approximate molecular weight.

6. The pharmaceutical composition according to claim 1, wherein the 6-TG and polyethylene oxide polymer are in a ratio of about 1:20 to 1:50.

7. The pharmaceutical composition according to claim 1, wherein the composition is a tablet comprising a core and an enteric coating.

8. The pharmaceutical composition according to claim 7, wherein the tablet comprises about 1 to 50 mg of 6-TG.

9. The pharmaceutical composition according to claim 1, wherein the polyethylene oxide polymer comprises about 60 to 98% w/w of the pharmaceutical composition.

10. The pharmaceutical composition according to claim 7, wherein the polyethylene oxide polymer comprises about 70 to 99% w/w of the core of the tablet.

11. The pharmaceutical composition according to claim 7, wherein the enteric coating comprises cellulose acetate phthalate, hydroxy propyl methyl cellulose acetate succinate, a methacrylic acid copolymer, or combinations thereof.

12. The pharmaceutical composition according to claim 11, wherein the enteric coating further comprises one or more excipients selected from: a plasticiser, an anti-tacking agent, a surfactant, an antifoam agent, and mixtures thereof.

13. The pharmaceutical composition according to claim 7, wherein the enteric coating is capable of dissolving at a pH of at least 5.5.

14. The pharmaceutical composition according to claim 1, wherein the pharmaceutical composition is formulated to provide approximately zero-order kinetics of 6-TG release.

15. The pharmaceutical composition according to claim 1, wherein the pharmaceutical composition provides release of at least about 30% of the 6-TG in vitro at about pH 5.5 to about pH 7.5 by approximately 10 hours.

16. The pharmaceutical composition according to claim 1, wherein the pharmaceutical composition provides release of at least 70% of the 6-TG in vitro at about pH 5.5 to about pH 7.5 by approximately 20 hours.

17. (canceled)

18. The pharmaceutical composition according to a claim 1, wherein the pharmaceutical composition provides release of less than about 10% of the 6-TG in vitro at less than about pH 5.5 by approximately 2 hours.

19. The pharmaceutical composition according to claim 1, wherein the pharmaceutical composition provides release of less than 80% of the 6-TG in vitro at about pH 5.5 to about pH 7.5 by approximately 10 hours.

20. The pharmaceutical composition according to claim 1, wherein the pharmaceutical composition provides release of:
at least 85% of the 6-TG in vitro at about pH 5.5 to about pH 7.5 by approximately 30 hours, and
less than 80% of the 6-TG in vitro at about pH 5.5 to about pH 7.5 by approximately 10 hours.

21. A method for treating a disease or condition of the distal intestine that responds to 6-TG in an individual in need thereof, the method comprising administering the pharmaceutical composition according to claim 1, wherein the 6-TG is released in the distal intestine.

22. (canceled)
